(19) United States
(12) Patent Application Publication
(10) Pub. No.: US 2012/0171194 A1
(43) Pub. Date: Jul. 5, 2012

(54) Method of Cell Differentiation and Uses Therefor in Screening, Diagnosis and Therapy

(76) Inventor: Colin Tso, West Pymble (AU)

(21) Appl. No.: 13/257,047

(22) PCT Filed: Mar. 8, 2010
(86) PCT No.: PCT/AU2010/000251

§ 371 Date: Mar. 16, 2012

(30) Foreign Application Priority Data
Mar. 17, 2009 (AU) 2009901166
Oct. 23, 2009 (AU) 2009905195

(51) Int. Cl. A61K 39/395; C12Q 1/04; G01N 21/64; A61P 9/00; A61K 38/02; A61K 48/00; A61P 9/10; C12N 5/02; A61K 38/17

(52) U.S. Cl. 424/130.1; 435/377; 435/34; 435/7.1; 514/21.2; 514/1.9; 514/44 R

(57) ABSTRACT

The present invention provides a method of producing non-haematopoietic cells, in particular cells having one or more properties of endothelial cells, from monocytes. A range of diagnostic and prognostic assay methods for determining whether or not a subject has atherosclerosis or vascular complication associated therewith, or a predisposition for developing atherosclerosis or complication associated therewith employing this method are provided. A range of screens to identify and isolate compositions of matter that modulate the ability of monocytes to differentiate into such non-haematopoietic cells and/or that modulate the ability of such non-haematopoietic cells to become incorporated into endothelium e.g., vascular endothelium, are also provided.

### FIELD OF THE INVENTION

The present invention relates to the field of cell biology, drug screening, diagnosis and therapeutics, such as for vascular processes.

### BACKGROUND OF THE INVENTION

### Atherosclerosis

Atherosclerosis is the leading cause of death in western industrialized countries. Atherosclerosis is characterized in part by an inflammatory response in the walls of arteries and/or narrowing of the arterial lumen e.g., due to the accumulation of lipid-laden macrophages such as foam cells within the arteries thereby forming atherosclerotic plaques. The macrophages accumulate lipids through the ingestion of low-density lipoproteins (LDL) that transport cholesterol. Without adequate removal of fats and cholesterol from the macrophages, atherosclerotic plaques enlarge under the inner lining of the arteries e.g. the endothelium. Atherosclerosis is triggered e.g., by injury to the endothelium that lines the entire inner wall of the arterial tree. For example, monocytes are recruited to the endothelium, where they adhere, transmigrate into the arterial wall and differentiate into macrophages. Monocytes differentiation into macrophages is traditionally thought to be the exclusive outcome of monocytic adherence to the endothelium. The risk of developing atherosclerosis is directly related to the plasma levels of LDL and inversely related to another fraction of plasma cholesterol known as high-density lipoproteins (HDL).

The complications of atherosclerosis arise from the impediment to arterial blood flow due to the intrusion of atherosclerotic plaques into the arterial lumen, ultimately leading to tissue damage. This process can either be chronic, slowly progressive luminal narrowing, or acute blockage due to sudden rupture of the atherosclerotic plaques with resultant thrombus formation. Insufficient blood supply to an organ or tissue fed by the blocked or narrowed artery will then lead to cell death, tissue necrosis and organ failure such as myocardial infarction. Conversely, atherosclerotic plaques and luminal narrowing may be compensated by artery enlargement, resulting in aneurysmal formation.

### Vascular Disease

Vascular disease is the leading causes of morbidity and mortality in Western Societies. In practice, vascular injuries accumulate from adolescence prior to the full manifestation of the disease. By the time the vascular complications are detected, the underlying cause is usually quite advanced, having progressed for decades, making primary diagnosis and prevention efforts necessary from early stages of childhood.

As used herein, the term “vascular disease” or “vascular condition” shall be taken to mean a disease or condition characterised by one or more impairment(s) associated with the vasculature or the vascular tissue and/or affecting the vasculature or the vascular tissue. Examples of vascular disease include but are not limited to cardiovascular disease, ischaemia, and diabetes.

### 1. Cardiovascular Disease

Cardiovascular disease accounts for one in every two deaths in the United States and most European countries as well as Australia and New Zealand. Two out of three cardiac deaths occur without any prior diagnosis of cardiovascular disease. Thus, diagnosis and prevention of cardiovascular disease is an area of major public health importance.

Atherosclerosis is the major cause of cardiovascular disease. Cardiovascular disease is a general term encompassing diseases of the circulatory system, e.g., atherosclerosis, coronary heart disease, cerebrovascular disease, aorto-iliac disease, or peripheral vascular disease. Subjects with cardiovascular disease may develop a number of complications, including, but not limited to, myocardial infarction, stroke, angina pectoris, transient ischemic attacks, congestive heart failure, aortic aneurysm and death.

### 2. Ischemia

“Ischemia” or “Ischemic disease” is a general term encompassing conditions in which blood flow and thus delivery of oxygen is restricted to a part of the body or a particular organ. Ischemia encompasses but not limited to Ischemic heart disease (IHD), or myocardial ischaemia, which occurs due to the lack of blood flow and oxygen to the heart muscle because of coronary artery disease e.g., atherosclerosis of the coronary arteries. In general, tissue ischemia is caused by narrowing of the arteries. When arteries are narrowed, less blood and oxygen reaches the organ, e.g., the heart, this leads to damage of the heart muscle. Coronary artery disease is progressive, ultimately leading to heart damage. For example, the risk of developing IHD increases with age, smoking, hypercholesterolaemia (high cholesterol levels), diabetes, hypertension (high blood pressure) and is more common in men and those who have close relatives with ischaemic heart disease.

### 3. Diabetes and Vascular Complications Thereof

Diabetes is characterized by one or more of these conditions: impaired glucose tolerance, insulin resistance, and hyperglycemia e.g., including diabetes mellitus. Examples of diabetes in this context include Type 1 diabetes and Type 2 diabetes.

Diabetes afflicts an estimated 194 million people worldwide e.g., approximately 7.9% of Americans and approximately 7.8% of Europeans. Effective clinical management of diabetes is recognized as a growing problem, especially in societies where diet contributes to the growth of Type 2 diabetes e.g., Nesto, Rev. Card. Med. 4, S11-S18 (2003) the disclosure of which is hereby incorporated by reference in its entirety. Between about 85% and 95% of all diabetics suffer from Type 2 diabetes, although nearly 5 million people worldwide suffer from Type 1 diabetes, affecting an estimated 1.27 million people in Europe and about 1.04 million people in the United States.

Type 1 diabetic patients and Type 2 diabetic patients are each at higher risk than non-diabetic subjects for a wide array of vascular complications including microvascular and macrovascular complications. Microvascular complications include, for example, neuropathy, retinopathy (e.g., diabetic retinopathy), nephropathy (e.g., diabetic kidney disease), impotence, and impaired or slow wound healing e.g., of ulcers. Macrovascular complications include, for example, extracranial and intracranial vascular disease (e.g., contributing to ischemia and/or stroke), coronary heart disease, and peripheral vascular disease.

Neuropathy results in a loss of sensation in the peripheral limbs e.g., feet, exposing patients to undue, sudden or repetitive stress. Neuropathy can cause a lack of awareness of damage to limbs, and/or a risk of fissures, creating a potential entry for bacteria and increased risk of infection.

The peripheral vasculature of a diabetic patient may result in die foot that affects the ability of a patient to walk and/or stand. Diabetic feet are at risk for a wide range of pathologies, including peripheral vascular disease, microcirculatory changes, neuropathy, ulceration, infection, deep tissue destruction and metabolic complications. The development of an ulcer in the diabetic foot is commonly a result of a break in the barrier between the dermis of the skin and the subcutaneous fat that cushions the foot during ambulation. This, in turn, can lead to increased pressure on the dermis, resulting in tissue ischemia and eventual necrosis and/or apoptosis, and ulceration. Progressive peripheral vascular disease in diabetic subjects may result in infection leading to gangrene, which is generally treated surgically by amputation. A foot ulcer precedes roughly 85% of all lower extremity amputations in diabetic patients, and about 15% of all diabetic patients will develop a foot ulcer during the course of their lifetimes. Infected and/or ischemic diabetic foot ulcers account for about 25% of all hospital days among people with diabetes, and the costs of foot disorder diagnosis and management are estimated at several billion dollars annually.

Diabetic retinopathy is an eye disease associated with diabetes wherein damage is caused to the blood vessels feeding the retina. Every person with diabetes will develop some degree of diabetic retinopathy which, without treatment may cause loss of vision and ultimately, blindness. Diabetic subjects are about 25 times more likely to lose vision or go blind than non-diabetic subjects. Temporal progression of diabetes increases the risk of diabetic retinopathy, however there may be no obvious symptoms of retinopathy in the early stages of diabetes e.g., in juveniles. In the early stages of diabetic retinopathy, there are also no apparent symptoms, and vision may not become impaired until the disease has progressed. There are two forms of retinopathy that can affect sight: (a) maculopathy, that develops when some of the small blood vessels around the macula become occluded, thereby causing other blood vessels to leak such that fluids build up around the macula causing swelling and loss of sight; and (b) proliferative retinopathy, that develops when blood vessels develop and/or grow abnormally on the surface of the retina e.g., into the centre of the eye, wherein the abnormal blood vessels may bleed, thereby impairing vision. In proliferative retinopathy, healing of blood vessels that have bled may result in scar tissue formation, thereby increasing surface tension of the retina resulting in retinal detachment, loss of vision or blindness.

Diabetic nephropathy is characterized by hyperproteinurea and it is believed that uncontrolled high blood sugar leads to the development of kidney damage. High blood sugar levels may damage the multiple tiny blood vessels or nephrons of the kidney, which help remove waste from the body. This may cause the nephrons to thicken and become scarred, and gradually they may become destroyed. Eventually this damage may cause the kidneys to leak and protein, including albumin, begins to pass into the urine.

It has been estimated that about 35-75% of men with diabetes may experience at least some degree of erectile dysfunction, or impotence, during their lifetime, which may be developed 10 to 15 years earlier than in healthy non-diabetic men. Erectile dysfunction in men with diabetes is likely to be caused by impairments in nerve, blood vessel and muscle function. To get an erection, men need healthy blood vessels, nerves, male hormones, and a desire to be sexually stimulated. However, diabetes may lead to damage of the blood vessels and nerves that control erection. Therefore, a diabetic male may not be able to achieve a firm erection, even if an adequate amounts of male hormones and sexual stimulation are present.

Patients with diabetes often have wounds that are difficult to heal. Hyperglycemia, or an increased blood glucose level, is often the initial barrier to wound healing, such as ulcers, including foot ulcers, in diabetic subjects. The increased blood glucose level, may cause the cell walls to become rigid, impairing blood flow through the critical small vessels at the wound surface and impeding red blood cell permeability and flow. This impairs hemoglobin release of oxygen, which results in oxygen and nutrient deficits in the wound. Persistently elevated blood glucose levels may lead to compromise chemotaxis and phagocytosis immune functions, which also contributes to poor wound healing in subjects suffering from hyperglycemia. Chemotaxis is the process by which white cells are attracted to the site of an infection, and phagocytosis is the ingestion of bacteria by white cells. Both chemotaxis and phagocytosis are important in controlling wound infections. Diabetic infections take longer to heal because of delayed macrophage introduction and diminished leukocyte migration, which causes a prolonged inflammatory phase in the wound-healing cascade.

The macrovascular complications of diabetes mellitus are characterised by accelerated atherosclerosis, leading to more aggressive coronary artery disease, cerebrovascular disease and peripheral vascular disease. Coronary heart disease involves a failure of coronary circulation to supply adequate circulation to cardiac muscle and surrounding tissue. Peripheral vascular disease (PAD) refers to diseases of blood vessels outside the heart and brain. It's often a narrowing of vessels that carry blood to the legs, arms, stomach or kidneys. Peripheral vascular disease may have short-term effects that do not involve defects in blood vessels' structure, or may be caused by structural changes in the blood vessels, such as inflammation and tissue damage. People with peripheral vascular disease often have atherosclerosis of other parts of the vasculature including the heart and brain. Because of this association, most people with PAD have a higher risk of death from heart attack and stroke. Chronic hyperglycemia, such as that in diabetic subjects, increases the risk macrovascular complications.

Notwithstanding the associations between vascular diseases such as those manifested in diabetes patients and increased risks of atherosclerosis and poor outcomes following vascular occlusion, the precise mechanisms of vascular maintenance or vascular repair or vascular regeneration are poorly understood.

### Vascular Endothelium

Vascular endothelial cells (VECs) cover the entire inner surface of blood vessels in the body. They play an important role in tissue homeostasis, fibrinolysis and coagulation, blood-tissue exchange, vascularization of normal and neoplastic tissues, and blood cell activation and migration during physiological and pathological processes.

Endothelial health reflects a balance between injury and repair, and is a key component of many vascular diseases or conditions. Vascular endothelium acts as a potent anti-thrombotic, antioxidant and anti-inflammatory barrier. These functions are critical in protecting the vessel wall from developing pathologies such as atherosclerosis. Accordingly, prolonged and repeated exposure to adverse conditions may cause injury to the endothelium. Such adverse conditions include but are not limited to oxidative stress and chronic inflammation, which are intimately associated with cardiovascular risk factors such as hypercholesterolemia, hyperglycemia, hypertension, low shear stress and smoking ultimately blunts these protective mechanisms. These conditions inter alia, may render the endothelium dysfunctional and increased apoptosis and subsequent detachment of the endothelium from the underlying intimal layer. Dysfunctional vascular endothelium is associated with increase in proliferation of vascular smooth muscle cells, enhanced recruitment of monocytes as precursor or macrophages, lipid deposition and inflammation, ultimately leading to atherosclerosis and vascular complications.

### Vascular and Tissue Repair Processes

Notwithstanding the association between endothelial damage and/or dysfunction and increased risk of atherosclerosis and/or vascular complications, the precise mechanisms that promote or enhance vascular and tissue repair remain poorly understood. To date only few vascular regenerative mechanisms have been investigated including angiogenesis, neovascularisation and endothelial repair of the vasculature.

Angiogenesis and neovascularization are physiological processes of new vessel formation and/or growth. Angiogenesis is a process by which new blood vessels or vascular networks are formed from extant blood vessels and/or the enlargement of pre-existing blood vessels. Neovascularization is a process by which new blood vessels or vascular networks are formed in tissues not processing extant blood vessels.

Angiogenesis normally occurs in a carefully controlled manner during embryonic development, during growth, and in special cases such as wound healing and the female reproductive cycle e.g., Wilting and Christ, Naturwissenschaften 83:153-164 (1996); Goodger and Rogers, Microcirculation 2:329-343 (1995); Augustin et al., Am. J. Pathol. 147(2):339-351 (1995). However, this is also a fundamental step in the transition of tumors from a dormant state to a malignant state. In an adult, two types of blood vessels can potentially be found. The normal blood vessel is a resting, quiescent, fully developed vessel. A second form, a proliferating or developing blood vessel, occurs rarely during the normal human life cycle (occurring only in early development and during reproduction, e.g., menstrual cycle and pregnancy). In contrast, the process of angiogenesis, the proliferation and development of new blood vessels, often occurs in wound healing and in pathological processes, e.g., tumor growth. In pathological angiogenesis improper control of angiogenesis causes either excessive or insufficient blood vessel growth. Excessive blood vessel proliferation in cancer-related conditions favours tumor growth and development of distant metastases. In other diseases, it is the root cause of tissue injury, including blindness associated with proliferative retinopathies, diabetic retinopathy, and skin disorders such as psoriasis, and rheumatoid arthritis, e.g., Folkman, Nature Med. 1(1):27-31 (1995); Polverini, Rheumatology 38(2):103-112 (1995); Healy et al., Hum. Reprod. Update 4(5):736-396 (1998).

Angiogenesis is a complex process involving many stages, including extracellular matrix remodeling, migration proliferation and tubulogenesis of endothelial cells (ECs), capillary differentiation, and anastomosis. Some of the important steps in the process of angiogenesis are: 1) growth factor (e.g. vascular endothelial growth factor, VEGF) signaling; 2) matrix metalloproteinases (MMP) and VEGF receptor interaction; 3) endothelial cell (EC) migration e.g., VEC migration to site of growth factor signaling; and 4) tubule formation from ECs such as VECs. In general, angiogenesis occurs as diseased or injured tissues produce and release angiogenic growth factors that diffuse into the nearby tissues. The angiogenic growth factors activate ECs of nearby pre-existing blood vessels, to proliferate and migrate from the existing vessel towards the diseased or injured tissue. In this process, ECs become detached from the basement membrane as this support is degraded by proteolytic enzymes. These cells then migrate out from the parent vessel, divide, and form into a newly differentiated vascular structure, e.g., Risau, Nature 386:671-674 (1997); Wilting et al., Cell. Mol. Biol. Res. 41(4):219-232 (1995). Consequential vessel extension and remodeling ensue. Finally, newly formed blood vessel tubes are stabilized by specialized muscle cells e.g., smooth muscle cells, pericytes, that provide structural support. Blood flow then begins.

Proliferation of neighbouring ECs is traditionally considered to be the exclusive source of replacing damaged cells during vascular repair and regeneration. However, in work leading up to the present invention the inventor has shown that contrary to conventional belief endothelial progenitor cell is a major source of endothelial cell turnover in response to vascular injury.

### SUMMARY OF THE INVENTION

### 1. Introduction

In works leading up to the present invention the inventor sought to elucidate one or more cellular mechanisms involved in the rapid or early formation of mature ECs that contribute to regeneration of vasculature and/or promotion or enhancement of vascular repair processes. For example, the inventors sought to elucidate early stage events that contribute to endothelial maintenance and/or repair, in particular, the contribution of monocytes

As exemplified herein, the inventor has shown that monocytes brought into contact with ECs rapidly differentiate into non-haematopoietic cells, e.g., ECs or EC-like cells having one or more properties of mature ECs, suggesting that under certain conditions monocytes may participate in vascular repair by virtue of such trans-differentiation process. For example, the inventor has also demonstrated that monocytes differentiated into ECs or endothelial cell-like (“EC-like”) cells and that these monocyte-derived ECs and/or EC-like cells may become incorporated into endothelium.

As used herein, the term “endothelium” as it is understood in the art, shall be construed in its broadest context to include naturally occurring endothelium tissue in vivo or ex vivo, a monolayer of ECs and/or EC-like cells, a confluent or sub-confluent culture of ECs and/or EC-like cells. It is understood that the “endothelium” also includes proliferating and/or dividing and/or growing ECs and/or EC-like cells and may also includes contact inhibited and/or resting ECs and/or EC-like cells. Also it is understood that the “endothelium” does not include endothelial progenitor cells (EPCs), and does not include cells that express EPC cell surface markers such as CD133.

The inventor has also demonstrated that the differentiation and recruitment of the monocyte-derived EC or EC-like cells into the endothelium occurs rapidly within about 4 days, preferably within about 48 hours, more preferably within 24 hours after contact of monocytes with ECs. Such rapid differentiation and incorporation provides an index for quantifying the ratio of monocyte-derived ECs or EC-like cells relative to substrate monocytes, and/or relative to substrate ECs.

The term “monocyte” as it is understood in the art, shall be construed in its broadest context to mean a circulating mononuclear cell of myeloid lineage that expresses the cell surface markers including but not limited to CD14, CD11b (syn. Mac-1), CD31, ICAM-1 and/or CD36 but does not express the cell surface markers including but not limited to CD34, KDR (also known as VEGF-R2), Tie-2, CD144, CD105, CD146 (syn. P1H12). Also it is understood that a “monocyte” does not express macrophage or dendritic cell surface markers such as CD115 or CD16. Also it is understood that a “monocyte” does not encompass endothelial progenitor cells (EPCs), and does not express EPC cell surface markers such as CD133. When functional or normal, a monocyte is capable of differentiating into a macrophage. A monocyte may be functional, non-functional, or have impaired function e.g., with respect to this proliferative ability and developmental capacity, and non-functional monocyte having impaired function may be identified readily e.g., by its aberrant morphology and/or proliferative ability.

The term “non-haematopoietic cell” as it is understood in the art, shall be construed in its broadest sense to mean a cell that does not form a blood cellular component and is not derived from haematopoietic stem cell or haematopoietic progenitor cell, and/or has been derived by a cell lineage other than the erythroid lineage, lymphoid lineage or myeloid lineage.

The term “endothelial cell” or “EC” or “endothelial-like cell” or “EC-like cell” as it is understood in the art, shall be construed in its broadest sense to refer to a non-haematopoietic cell capable of forming the endothelium or an endothelial tissue. An endothelial cell” or “EC” or “endothelial-like cell” or “EC-like cell” is understood to expresses the cell surface markers including but not limited to CD105, CD144, CD31, ICAM-1, VCAM-1, CD146 but does not express the cell surface markers including but not limited to CD14, CD11b and CD36. Also it is understood that an “endothelial cell” or “EC” or “endothelial-like cell” or “EC-like cell” does not encompass endothelial progenitor cells (EPCs), and does not express EPC cell surface markers such as CD133.

Proceeding on this basis, the inventor reasoned that peripheral monocytes may provide an abundant source of non-haematopoietic cells for rapid vascular regeneration and/or vascular maintenance and/or vascular repair, including any ECs or EC-like cells as hereinbefore defined.

The inventor has further demonstrated that incorporation of monocyte-derived ECs is enhanced by factors or substrates of high-density lipoprotein (HDL), e.g., apolipoprotein A-I and/or apolipoprotein A-II, thereby implicating HDL or factors or substrates thereof in promoting endothelial and vascular maintenance and/or vascular repair processes.

The data herein are consistent with assays for trans-differentiation of monocytes into EC or EC-like cells. For example, factors in a subject's monocytes or circulation may affect the ability of the monocytes to undergo trans-differentiation into EC or EC-like cells. Proceeding on this basis, assays for differentiation of EC or EC-like cells from monocytes in the presence of a patient sample form the basis of a wide range of diagnostics and/or prognostic tests for determining the likelihood of a subject having or is at a risk of developing one or more vascular complications and/or atherosclerosis. Naturally such assays also have utility of monitoring vascular therapy.

By “vascular therapy” is meant any therapy for promoting or augmenting vascular maintenance and/or vascular repair and/or angiogenesis and/or neovascularisation including therapy for promoting or augmenting vascular network formation and/or development.

Similarly, assaying for the differentiation of monocytes into EC or EC-like cells has utility for identifying compositions of matter that enhance or promote and/or antagonise monocytes differentiation into non-haematopoietic cells.

Such modulatory compounds are useful to produce formulations, e.g., topical, injectable and oral formulations comprising the modulatory compositions of matter, for prophylactic and/or therapeutic intervention of atherosclerosis and/or one or more vascular complications associated with one or more vascular diseases and/or enhancing vascular repair, e.g., before the onset of atherosclerosis and/or one or more vascular complications in a subject.

It is to be understood, that in the context of prophylactic intervention, a subject for treatment will generally be identified by one or more risk factors including e.g., ischemia, myocardial infarct, hypertriglyceridemia, hypercholesteremia, hyperglycemia, hypertension, smoking, Type 1 diabetes, Type 2 diabetes, increased age.

For therapeutic interventions of existing vascular complications, the subject may be identified by one or more risk factors supra if the vascular complication(s) exist at an early stage, that is not readily apparent, or by other means e.g., impaired angiogenic function and/or impaired neovascularization, aberrant migration of ECs, proliferation of ECs, tubulogenesis of ECs or combination thereof. Thus vascular complications that are apparent in a subject may be identified by the vascular complication per se and/or one or more risk factors or other means supra.

Accordingly, the term “vascular complications” shall be taken to mean any one or more or a combination of vascular complications associated with a vascular disease or vascular condition for example including but not limited to cardiovascular disease, ischaemia, and diabetes. Examples of vascular complications include but are not limited to, injury and/or damage to the endothelium, a microvascular or macrovascular complication and/or impaired angiogenesis and/or impaired neovascularization, including e.g., neuropathy, retinopathy (e.g., diabetic retinopathy), nephropathy (e.g., diabetic kidney disease), impotence, impaired or low wound heeling e.g., of ulcers, extracranial vascular disease, intracranial vascular disease, coronary heart disease, peripheral vascular disease, myocardial infarction, stroke, angina pectoris, transient ischemic attacks, congestive heart failure, aortic aneurysm, ischaemia disease and combinations thereof.

The present invention clearly encompasses the production and use of EC-like cells for promoting or augmenting vascular maintenance and/or vascular repair and/or angiogenesis and/or neovascularisation.

As used herein, “vascular maintenance” is to be understood in its broadest sense to mean the maintenance of vascular cells in existing vasculature by a process involving EC and/or comprising EC-like cell migration, proliferation and mobilisation.

As used herein, “vascular repair” or the similar term “vascular regeneration” is to be understood in its broadest sense to mean a turnover of damaged vascular cells in existing vasculature by a repair process involving EC and/or comprising EC-like cell migration, proliferation and mobilisation.

As used herein, the term “angiogenesis” shall be taken to mean a process by which new blood vessels are formed whether from extant blood vessels or not and/or the enlargement of pre-existing blood vessels by a mechanism involving EC and/or comprising EC-like cell migration, proliferation and mobilisation.

As used herein, the term “neovascularization” shall be taken to mean a development of new blood vessels in tissues not possessing extant blood vessels by a mechanism involving EC and/or comprising EC-like cell migration, proliferation and mobilisation.

Neovascularization in adults is thought to be a mixed phenomenon of angiogenesis and vasculogenesis. Vasculogenesis is understood to be the differentiation of endothelial progenitor cells or stem cells or cells capable of adopting an endothelial lineage to ECs and subsequent formation of primary vascular plexus.

### 2. Specific Embodiments

The scope of the invention will be apparent from the claims as filed with the application that follow the examples. The claims as filed with the application are hereby incorporated into the description. The scope of the invention will also be apparent from the following description of specific embodiments.

In one example, the present invention provides a method of differentiating monocytes into non-haematopoietic cells, said method comprising contacting substrate monocytes with substrate ECs for a time and under conditions sufficient for non-haematopoietic cells to be produced from the substrate monocytes. For example, the produced non-haematopoietic cells are ECs and/or EC-like cells expressing CD144 and/or CD105.

In one example, the substrate monocytes and produced non-haematopoietic cells may express the same alleles of a detectable marker and wherein substrate ECs cells may express one or more different alleles of the detectable marker to thereby distinguish the substrate ECs from the substrate monocytes and from the produced non-haematopoietic cells.

Also in accordance with this example, the method may further comprise determining expression of one or more different alleles of the detectable marker in the substrate monocytes and/or the produced non-haematopoietic cells and/or the substrate ECs, wherein expression of different alleles of the detectable marker in the substrate ECs to the alleles of the detectable marker expressed by the substrate monocytes and produced non-haematopoietic cells is indicative of non-haematopoietic cell production from the monocytes. In one example, the method may comprise determining expression of the detectable marker in the substrate monocytes and the substrate ECs. In another example, the method may comprise determining expression of the detectable marker in the substrate ECs and in the produced non-haematopoietic cells. In yet another example, the method may comprise determining expression of the detectable marker in the substrate monocytes and in the substrate ECs and in the produced non-haematopoietic cells.

Preferably, the detectable marker is a cell surface antigen. More preferably, the detectable marker is a human leukocyte antigen.

In one example, the method may further comprise quantitating production of non-haematopoietic cells from substrate monocytes. Preferably, quantitating production of non-haematopoietic cells from substrate monocytes comprises determining a ratio or percentage or number of substrate monocytes that differentiate into non-haematopoietic cells. In accordance with this example, the substrate monocytes and/or produced non-haematopoietic cells are determined by the presence or absence of one or more surface cell-type specific markers. Preferably, a surface cell-type specific marker is a cluster designation (CD) marker, e.g., a marker selected from the group consisting of CD14, CD11b, CD115, CD34, CD105, CD133, CD144, Tie-2 KDR, ICAM-1 and VCAM-1, and preferably selected from CD14, CD11b, CD144, CD105, CD133, Tie-2, KDR, CD34, ICAM-1, VCAM-1, CD115, CD146 and CD36.

Preferably, the time sufficient for non-haematopoietic cells to be produced is less than about 4 days, or less than about 3 days, or less than about 2 days (i.e., 48 hours), or about 24 hours.

Cells such as the substrate monocytes and substrate ECs used in the method of the invention, are generally, but not necessarily allogeneic (i.e., isolated from the same species). For example, the cells may be isolated from a healthy subject i.e., a subject not suffering from one or more vascular complications and/or atherosclerosis and/or not at a risk of suffering from one or more vascular complications and/or atherosclerosis, and optionally expanded. Allogeneic cells may also be autologous, i.e., derived from the same subject. If the cells are autologous, they may be isolated from the subject, optionally genetically repaired or otherwise treated ex vivo by any means known in the art to render them functional, and then optionally expanded. Alternatively, functional cells are isolated from a subject to be treated, expanded ex vivo and used in the method of the invention.

The use of xenogeneic cells is also encompassed by the invention and the invention particularly contemplates the use of porcine or primate e.g., orang-utan monocytes substrates with human EC substrates and vice versa the use of porcine or primate e.g., orang-utan substrate ECs with substrate human monocytes.

In a further example, the method according to the invention is used for determining the ability of a composition to produce non-haematopoietic cells or to promote the production of non-haematopoietic cells. Alternatively, or in addition, the method according to the invention is used for determining the ability of a composition to antagonize the production of non-haematopoietic cells.

In accordance with this example, the composition may comprise substrate monocytes and the method according to the invention may comprise determining production of non-haematopoietic cells from the substrate monocytes in the composition.

In one example, the method according to the invention may comprise determining the ability of a composition to promote the production of non-haematopoietic cells. In accordance with this example, the composition may lack monocytes or may be monocytes-depleted and the method according to the invention may comprise contacting exogenous substrate monocytes with the substrate ECs in the presence and absence of the composition for a time and under conditions sufficient for non-haematopoietic cells to be produced from the substrate monocytes and comparing non-haematopoietic cell production, wherein enhanced non-haematopoietic cell production in the presence of the composition indicates that the composition promotes the production of non-haematopoietic cells.

In another example, the method according to the invention may comprise determining the ability of a composition to antagonize the production of non-haematopoietic cells. In accordance with this example, the composition may lack monocytes or may be monocyte-depleted and the method according to the invention may comprise contacting exogenous substrate monocytes with the substrate ECs in the presence and absence of the composition for a time and under conditions sufficient for non-haematopoietic cells to be produced from the substrate monocytes and comparing non-haematopoietic cell production, wherein reduced non-haematopoietic cell production in the presence of the composition indicates that the composition antagonizes the production of non-haematopoietic cells.

In a further example, the method of the present invention encompasses screening for, isolating and/or identifying a modulator of angiogenesis and/or neovascularization and/or atherogenesis and/or a vascular complication.

In a further example, the method of the present invention encompasses determining the likelihood of a subject having one or more vascular complications and/or atherosclerosis and/or at risk of developing one or more vascular complications and/or atherosclerosis. According to this example, said method may be performed in the presence of a sample obtained from the subject.

In a further example, the method of the present invention encompasses monitoring therapy of a subject for one or more vascular complications and/or atherosclerosis, wherein said method is performed in the presence of two or more samples obtained from the subject at different time points.

In a further example, the present invention encompasses a process for determining the ability of a composition to modulate the production of non-haematopoietic cells from monocytes, comprising differentiating monocytes into non-haematopoietic cells according to the present invention in the presence of a candidate modulatory composition to thereby determine the effect of the candidate modulatory composition on non-haematopoietic cell production and identifying and/or isolating a composition that promotes the production of non-haematopoietic cells or antagonizes the production of non-haematopoietic cells.

Preferably, the process of determining the ability of a composition to modulate the production of non-haematopoietic cells from monocytes may be performed in vivo. In accordance with this example, the ability of a composition to modulate the production of non-haematopoietic cells from monocytes in vivo preferably comprises administering a candidate modulatory composition to an animal subject and determining the effect of the composition on atherogenesis and/or one or more vascular maintenance and/or repair processes in the animal subject and identifying and/or isolating a composition that promotes or antagonizes atherogenesis and/or one or more vascular maintenance and/or repair processes in the animal subject.

Alternatively, or in addition, the process of determining the ability of a composition to modulate the production of non-haematopoietic cells from monocytes may be performed in vitro. In accordance with this example, the candidate modulatory composition is preferably an agonist or partial agonist of non-haematopoietic cell production. Also in accordance with this example, the process of determining the ability of a composition to modulate the production of non-haematopoietic cells from monocytes preferably further comprises performing the method according to the invention in the absence of substrate ECs and comparing the effect of the agonist or partial agonist in the presence and absence of substrate ECs.

In accordance with this example, the process of determining the ability of a composition to modulate the production of non-haematopoietic cells from monocytes preferably comprises identifying and/or isolating a composition that promotes the production of non-haematopoietic cells in the absence of substrate ECs.

Also, in accordance with this example, the candidate modulatory composition may be an antagonist of non-haematopoietic cell production.

In another example, the process of determining the ability of a composition to modulate the production of non-haematopoietic cells from monocytes may further comprise differentiating monocytes into non-haematopoietic cells according to the invention in the presence of an agonist of EC production and comparing the effect of the antagonist on non-haematopoietic cell production in the presence and absence of the agonist. Preferably, the agonist of EC production comprises fibronectin and one or more growth factors under angiogenic culture conditions. Also preferably, the process of determining the ability of a composition to modulate the production of non-haematopoietic cells from monocytes may be performed in a single reaction in the presence of the agonist. Alternatively, the process of determining the ability of a composition to modulate the production of non-haematopoietic cells from monocytes may be performed in separate reactions in the presence and absence of the agonist.

In accordance with this example, the process of determining the ability of a composition to modulate the production of non-haematopoietic cells from monocytes may further comprise identifying and/or isolating a composition that antagonizes the production of non-haematopoietic cells in the presence of the agonist.

In a further example of the present invention, the process of determining the ability of a composition to modulate the production of non-haematopoietic cells from monocytes preferably further comprises differentiating monocytes into non-haematopoietic cells according to the present invention in the absence of the candidate modulatory composition and comparing non-haematopoietic cell production in the presence and absence of the candidate modulatory composition.

Preferably, the candidate modulatory composition preferably comprises a protein, polypeptide, peptide, antibody, nucleic acid, or small molecule.

Preferably, the process of determining the ability of a composition to modulate the production of non-haematopoietic cells from monocytes further comprising administering the isolated or identified composition to an animal subject and determining the effect of the composition on one or more vascular maintenance and/or repair processes in the animal subject.

In another example, a method of treatment or prophylaxis as described herein according to any example hereof additionally comprises providing or obtaining an amount of a composition as described herein above.

In one example, the process of determining the ability of a composition to modulate the production of non-haematopoietic cells from monocytes preferably further comprising performing one or more of the following:
i) determining the structure of an isolated or identified composition;
ii) providing the isolated or identified composition or the structure of the isolated or identified composition;
iii) synthesizing the isolated or identified composition; or
iv) obtaining the isolated or identified composition.

In a further example, the present invention encompasses a composition identified or isolated by the process of determining the ability of a composition to modulate the production of non-haematopoietic cells from monocytes, according to the present invention.

In a further example, the present invention also provides a process for determining the ability of a composition to promote the production of non-haematopoietic cells from monocytes, said process comprising providing the composition to a culture comprising substrate monocytes for a time and under conditions sufficient for non-haematopoietic cells to be produced from the substrate monocytes to thereby determine the effect of the candidate modulatory composition on non-haematopoietic cell production and identifying and/or isolating a composition that promotes the production of non-haematopoietic cells.

In one example, the present invention comprises performing a process according to any example hereof wherein the produced non-haematopoietic cells are ECs and/or EC-like cells expressing CD144 and/or CD105 and/or CD146.

In another example, the present invention comprises performing a process according to any example hereof wherein the candidate modulatory composition comprises a protein, polypeptide, peptide, antibody, nucleic acid, or small molecule.

In another example, the present invention comprises performing a process according to any example hereof wherein said process further comprises administering the isolated or identified composition to an animal subject and determining the effect of the composition on one or more vascular maintenance and/or repair processes in the animal subject.

In another example, the present invention comprises performing a process according to any example hereof wherein said process further comprises performing one or more of the following:
i) determining the structure of an isolated or identified composition;
ii) providing the isolated or identified composition or the structure of the isolated or identified composition;
iii) synthesizing the isolated or identified composition; or
iv) obtaining the isolated or identified composition.

Preferably, the term “isolating” comprises the use of any chemical or biochemical purification process known in the art to fractionate the mixture of plurality of compounds or compositions, coupled with assaying the fractions produced for activity with respect to monocyte differentiation into non-haematopoietic cells and/or vascular maintenance and/or repair activity, and selecting fractions having one or more of said activities.

More preferably, the term “isolating” refers to a process that comprises iterated use of any chemical or biochemical purification process known in the art to partially or completely purify a composition or compound from a mixture of plurality of compositions or compounds and assaying the fractions produced in each iteration of the process for activity with respect to monocytes differentiation into non-haematopoietic cells and/or vascular maintenance and/or repair, and selecting at each iteration one or more fractions having one or more of said activities. Preferably, the process is repeated for n iterations wherein n is sufficient number of iterations to reach a desired purity of the composition or compound e.g., 50% or 60% or 70% or 80% or 90% or 95% or 99%. More preferably, the process is repeated for zero to about ten iterations. As will be known to the skilled artisan, such iterations do not require iteration of precisely the same purification processes and more generally utilize different processes or purification conditions for each iteration.

In the case of a library of compositions or compounds displayed separately wherein each composition or compound is substantially pure prior to performance of the method, such isolation results in the separation of the composition or compound or from other compositions or compounds in the library that do not have the requisite activity. In this case, the term “isolating” extends to determining the activity of one library component relative to another library component and selecting a composition or compound having the desired activity.

The present invention clearly extends to the direct product of any method of identification or isolation of a compound or other composition of matter described herein. Accordingly, in a one example, the present invention encompasses a composition identified or isolated by the process according to any example hereof. In another example, the present invention encompasses a non-haematopoietic cells produced in accordance with the inventive method, e.g., monocyte-derived ECs and/or monocyte-derived EC-like cells, or vascular endothelium incorporating such cell products.

In a further example, the present invention provides a process of determining the likelihood of a subject having one or more vascular complications and/or atherosclerosis and/or at risk of developing one or more vascular complications and/or atherosclerosis, said process comprising differentiating monocytes into non-haematopoietic cells, e.g., ECs and/or EC-like cells, such as non-haematopoietic cells expressing CD144 and/or CD105, in the presence of a biological sample isolated from a subject, wherein production of non-haematopoietic cells from substrate monocytes in the presence of the biological sample indicates that the subject has a reduced or low likelihood of having said one or more vascular complications and/or atherosclerosis and/or is not at a risk of having or developing said one or more vascular complications and/or atherosclerosis, and/or wherein non-production or low production of non-haematopoietic cells from substrate monocytes in the presence of the biological sample indicates that the subject has one or more vascular complications and/or atherosclerosis and/or is at a risk of having or developing said one or more vascular complications and/or atherosclerosis. Preferably, the process further comprises obtaining a sample from a subject.

In accordance with this example, preferably the biological sample comprises the substrate monocytes and the method of differentiating monocytes into non-haematopoietic cells comprises determining the ability of the substrate monocytes to differentiate into non-haematopoietic cells e.g., ECs and/or EC-like cells, and wherein said differentiation indicates that the subject has a reduced or low likelihood of having said one or more vascular complications and/or atherosclerosis and/or is not at a risk of having or developing said one or more vascular complications and/or atherosclerosis, and/or wherein non-differentiation or a low efficiency of differentiation of said non-haematopoietic cells from substrate monocytes in the presence of the biological sample indicates that the subject has one or more vascular complications and/or atherosclerosis and/or is at a risk of having or developing said one or more vascular complications and/or atherosclerosis.

Also, in accordance with this example, preferably the sample comprises peripheral blood, umbilical cord blood, a fraction of peripheral blood comprising monocytes or a fraction of umbilical cord blood comprising monocytes. Preferably, a fraction of peripheral blood or a fraction of umbilical cord blood comprises plasma or fresh frozen plasma.

In another example, the sample lacks monocytes or is depleted of monocytes and preferably, said process comprises contacting the sample with substrate monocytes and determining the ability of the sample to promote differentiation of the substrate monocytes into non-haematopoietic cells e.g., ECs and/or EC-like cells, wherein said differentiation indicates that the subject has a reduced or low likelihood of having said one or more vascular complications and/or atherosclerosis and/or is not at a risk of having or developing said one or more vascular complications and/or atherosclerosis, and/or wherein non-differentiation or a low efficiency of differentiation of said non-haematopoietic cells from substrate monocytes in the presence of the biological sample indicates that the subject has one or more vascular complications and/or atherosclerosis and/or is at a risk of having or developing said one or more vascular complications and/or atherosclerosis.

In accordance with this example, preferably the sample lacking monocytes or depleted of monocytes is autologous to the substrate monocytes. Alternatively, the substrate monocytes are heterologous to the sample.

In accordance with this example, preferably the process further comprising depleting monocytes from a monocyte-containing sample. Preferably, depleting monocytes from the sample may be by a process comprising performing density gradient centrifugation and/or contacting the sample with a solid phase surface to thereby adhere monocytes and/or an antibody that binds CD14 for a time and under conditions sufficient to bind to CD14 on monocytes. Alternatively, or in addition, depleting monocytes from the sample may be by a process comprising performing density gradient centrifugation to thereby separate monocytes from plasma and retaining the plasma fraction. Alternatively, or in addition, depleting monocytes from the sample may be by a process comprising contacting the sample with a solid phase surface to thereby adhere monocytes and then retaining the unbound liquid fraction.

In accordance with this example, preferably the process comprising contacting the sample with an antibody that binds CD14 for a time and under conditions sufficient to bind to CD14 on monocytes and then removing monocytes. Preferably, the antibody that binds CD14 is immobilized on a solid substrate. Also preferably, the solid substrate comprises a particle.

Preferably, the monocyte-containing sample comprises peripheral blood, umbilical cord blood, a fraction of peripheral blood comprising monocytes or a fraction of umbilical cord blood comprising monocytes. Preferably, a fraction of peripheral blood or a fraction of umbilical cord blood comprises plasma or fresh frozen plasma.

In a further example, the present invention provides a process of determining the likelihood of a subject having one or more vascular complications and/or atherosclerosis and/or at risk of developing one or more vascular complications and/or atherosclerosis, said process comprising differentiating monocytes into non-haematopoietic cells e.g., ECs and/or EC-like cells (such as non-haematopoietic cells expressing CD144 and/or CD105), in the presence of a biological sample isolated from a subject to thereby produce said non-haematopoietic cells and determining the ability of the produced non-haematopoietic cells to incorporate into vascular tissue, wherein incorporation of non-haematopoietic cells into vascular tissue indicates that the subject has a reduced or low likelihood of having said one or more vascular complications and/or atherosclerosis and/or is not at a risk of having or developing said one or more vascular complications and/or atherosclerosis, and/or wherein non-incorporation or low efficiency of incorporation of non-haematopoietic cells into vascular tissue indicates that the subject has one or more vascular complications and/or atherosclerosis and/or is at a risk of having or developing said one or more vascular complications and/or atherosclerosis.

In accordance with this example, preferably the process further comprising obtaining the biological sample from the subject.

Also in accordance with this example, preferably, the biological sample comprises substrate monocytes and the production of non-haematopoietic cells e.g., ECs and/or EC-like cells from said substrate monocytes is determined.

According to another example, the sample may lack monocytes or is depleted of monocytes and wherein the production of non-haematopoietic cells e.g., ECs and/or EC-like cells, from substrate monocytes is determined in the presence of the sample. Preferably, the sample and substrate monocytes are autologous.

In another example, the ability of the produced non-haematopoietic cells e.g., ECs and/or EC-like cells to incorporate into vascular tissue according to any example hereof is preferably determined by migration and/or proliferation and/or tubulogenesis of non-haematopoietic cells. Also preferably, the ability of the produced non-haematopoietic cells e.g., ECs and/or EC-like cells to incorporate into vascular tissue according to any example hereof is determined by Matrigel assay.

In another example, the process further comprises determining the ability of the produced non-haematopoietic cells to incorporate into vascular tissue in the presence of an athero-protective compound or a cholesterol-lowering compound e.g., a statin or HDL, preferably wherein the athero-protective compound is a cholesterol-lowering substance. The term “athero-protective” is known in the art to mean a capability of protecting against atherosclerosis, whereas the term “cholesterol-lowering” is understood to mean a capability of reducing cholesterol e.g., LDL.

In another example, the process further comprises determining the ability of the produced non-haematopoietic cells e.g., ECs and/or EC-like cells to incorporate into vascular tissue in the presence and absence of an athero-protective compound. According to this example, preferably, the process further comprising comparing the incorporation of the produced non-haematopoietic cells into vascular tissue in the presence and absence of the athero-protective compound, wherein incorporation or in the presence but not absence of the athero-protective compound or enhanced incorporation in the presence of the athero-protective compound relative to incorporation in the absence of the athero-protective compound indicates that the subject is likely to have one or more vascular complications and/or atherosclerosis and/or is at a risk of having or developing said one or more vascular complications and/or atherosclerosis.

In one example, the athero-protective compound comprises statin.

Alternatively, the athero-protective compound comprises a high-density lipoprotein (HDL) or other protein(s) that mimic(s) HDL. In other examples, the athero-protective compound comprises apolipoprotein A-I and/or apolipoprotein A-II and/or apolipoprotein A-IV.

In a further example, the present invention provides a process of determining the likelihood of a subject having one or more vascular complications and/or atherosclerosis and/or at risk of developing one or more vascular complications and/or atherosclerosis, said process comprising differentiating monocytes into non-haematopoietic cells e.g., ECs and/or EC-like cells such as, for example, non-haematopoietic cells expressing CD144 and/or CD105 and/or CD146, and determining the ability of the produced non-haematopoietic cells to incorporate into vascular tissue in the presence of a biological sample isolated from a subject, wherein incorporation of non-haematopoietic cells into vascular tissue indicates that the subject has a reduced or low likelihood of having said one or more vascular complications and/or atherosclerosis and/or is not at a risk of having or developing said one or more vascular complications and/or atherosclerosis, and/or wherein non-incorporation or low efficiency of incorporation of non-haematopoietic cells into vascular tissue indicates that the subject has one or more vascular complications and/or atherosclerosis and/or is at a risk of having or developing said one or more vascular complications and/or atherosclerosis.

In accordance with this example, preferably the process further comprising obtaining the biological sample from the subject. Preferably, the sample comprises plasma, serum, a fraction of plasma or a fraction of serum. Preferably, the sample comprise high density lipoprotein (HDL). Preferably, the HDL comprises apolipoprotein A-I and/or apolipoprotein A-II and/or apolipoprotein A-IV.

Also in accordance with this example, preferably the sample and substrate monocytes are autologous and/or the sample and produced non-haematopoietic cells e.g., ECs and/or EC-like cells are autologous.

Also in accordance with this example, preferably the ability of the produced non-haematopoietic cells e.g., ECs and/or EC-like cells, to incorporate into vascular tissue is determined by migration and/or proliferation and/or tubulogenesis of the non-haematopoietic cells. Preferably, the ability of the produced non-haematopoietic cells to incorporate into vascular tissue is determined by Matrigel assay.

Also in accordance with this example, the process preferably further comprising determining the ability of the produced non-haematopoietic cells e.g., ECs and/or EC-like cells to incorporate into vascular tissue in the presence of an athero-protective compound, such as a cholesterol-lowering compound.

In another example, the process comprising determining the ability of the produced non-haematopoietic cells e.g., ECs and/or EC-like cells to incorporate into vascular tissue in the presence and absence of an athero-protective compound, such as a cholesterol-lowering compound.

According to this example, preferably the process further comprising comparing the incorporation of the produced non-haematopoietic cells e.g., ECs and/or EC-like cells into vascular tissue in the presence and absence of the athero-protective compound, or in the presence but not absence of the athero-protective compound wherein incorporation or enhanced incorporation in the presence of the athero-protective compound relative to incorporation in the absence of the athero-protective compound indicates that the subject is likely to have one or more vascular complications and/or atherosclerosis and/or is at a risk of having or developing said one or more vascular complications and/or atherosclerosis. Preferably, the athero-protective compound comprises statin. Alternatively, the athero-protective compound comprises a high-density lipoprotein (HDL) or mimics a HDL. Preferably, the athero-protective compound comprises apolipoprotein A-I and/or apolipoprotein A-II, and/or apolipoprotein A-IV.

In a further example, the present invention provides a process of monitoring vascular therapy of one or more vascular complications and/or atherosclerosis in a subject, or for monitoring vascular repair therapy, said process comprising differentiating monocytes into non-haematopoietic cells e.g., ECs and/or EC-like cells (such as non-haematopoietic cells expressing CD144 and/or CD105 and/or CD146), in the presence of two or more biological samples from a subject at different time points, wherein enhanced production of non-haematopoietic cells from substrate monocytes in the presence of a sample obtained at a later time point indicates that the subject has responded to vascular therapy or is undergoing vascular repair.

In accordance with this example, preferably at least one of the two or more samples is obtained from the subject prior to vascular therapy or vascular repair therapy, and at least one of the two or more samples is obtained from the subject after commencement of vascular therapy or vascular repair therapy and wherein enhanced production of non-haematopoietic cells e.g., enhanced EC production and/or enhanced EC-like cell production by a sample obtained from the subject after commencement of vascular therapy or vascular repair indicates effective therapy or vascular repair therapy

In a further example, the present invention provides a process of monitoring vascular therapy of one or more vascular complications and/or atherosclerosis in a subject, or for monitoring vascular repair therapy, said process comprising differentiating monocytes into non-haematopoietic cells (e.g., ECs and/or EC-like cells, such as cells expressing CD144 and/or CD105 and/or CD146) in the presence of two or more biological samples from a subject at different time points to thereby produce two or more batches of non-haematopoietic cells, and determining the ability of each of said two or more batches of non-haematopoietic cells to incorporate into vascular tissue, wherein enhanced incorporation of non-haematopoietic cells into vascular tissue in the presence of a sample obtained at a later time point indicates that the subject has responded to vascular therapy or is undergoing vascular repair.

In accordance with this example, preferably at least one of the two or more samples is obtained from the subject prior to vascular therapy or vascular repair therapy and at least one of the two or more samples is obtained from the subject after commencement of vascular therapy or vascular repair and wherein enhanced non-haematopoietic cell production by a sample obtained from the subject after commencement of vascular therapy indicates effective therapy or effective vascular repair therapy.

In a further example, the present invention provides a process of monitoring vascular therapy of one or more vascular complications and/or atherosclerosis in a subject, or for monitoring vascular repair therapy, said process comprising differentiating monocytes into non-haematopoietic cells (e.g., ECs and/or EC-like cells such as expressing CD144 and/or CD105 and/or CD146) and determining the ability of each of said non-haematopoietic cells to incorporate into vascular tissue in the presence of two or more biological samples from a subject at different time points, wherein enhanced incorporation of non-haematopoietic cells into vascular tissue in the presence of a sample obtained at a later time point indicates that the subject has responded to vascular therapy or is undergoing vascular repair.

In accordance with this example, preferably at least one of the two or more samples is obtained from the subject prior to vascular therapy or vascular repair therapy and preferably at least one of the two or more samples is obtained from the subject after commencement of vascular therapy or vascular repair and preferably enhanced non-haematopoietic cell production by a sample obtained from the subject after commencement of vascular therapy indicates effective therapy or vascular repair therapy.

In a further example, the present invention provides a process for the prevention or treatment of one or more vascular complications and/or atherosclerosis in a subject, or for promoting vascular repair, said process comprising determining the likelihood of a subject having one or more vascular complications and/or atherosclerosis and/or at a risk of developing one or more vascular complications and/or atherosclerosis according to any example hereof, and administering or recommending therapy effective to alleviate one or more vascular complications and/or atherosclerosis to a subject in need thereof.

In a further example, the present invention provides a process for the prevention or treatment of one or more vascular complications and/or atherosclerosis in a subject, said process comprising monitoring vascular therapy of one or more vascular complications and/or atherosclerosis in a subject or monitoring vascular repair therapy according to any example hereof, and administering or recommending therapy effective to alleviate one or more vascular complications and/or atherosclerosis to a subject in need thereof.

In a further example, the present invention encompasses use of the composition identified or isolated by a process of determining the ability of a composition to modulate the production of non-haematopoietic cells from monocytes according to any example hereof, for the treatment of one or more vascular complications and/or atherosclerosis in a subject in need thereof.

In a further example, the present invention provides a process for the prevention or treatment of one or more vascular complications and/or atherosclerosis in a subject, said process comprising administering to a subject in need thereof, a composition capable of modulating production of non-haematopoietic cells from monocytes according to any example hereof, identified or isolated according to any embodiment herein.

In another example, a method of treatment or prophylaxis as described herein according to any embodiment additionally comprises providing or obtaining an amount of a composition as described herein above. Accordingly, in one example, the present invention encompasses use of the composition identified or isolated by a process of determining the ability of a composition to promote the production of non-haematopoietic cells from monocytes according to any example hereof, for the treatment of one or more vascular complications and/or atherosclerosis in a subject in need thereof.

It is to be understood that an identified or isolated composition or compound in substantially pure form i.e., free from contaminants that might cause adverse side effects or contraindications or antagonize the activity of the active composition or compound, can be formulated into a medicament suitable for treatment of vascular complication(s) in hyperglycaemic and/or diabetic subjects. Accordingly, in one example, the present invention encompasses use the composition identified or isolated by a process of determining the ability of a composition to modulate the production of non-haematopoietic cells from monocytes according to any example hereof, in the preparation of a medicament for the treatment of one or more vascular complications and/or atherosclerosis in a subject in need thereof.

In a further example, the present invention encompasses use the composition identified or isolated by a process of determining the ability of a composition to promote the production of non-haematopoietic cells from monocytes according to any example hereof, in the preparation of a medicament for the treatment of one or more vascular complications and/or atherosclerosis in a subject in need thereof.

As used herein, the term “composition” or the term “compound” in its broadest scope shall be taken to mean a discrete chemical entity e.g., peptide, protein, nucleic acid, antibody, small molecule, or a plurality of discrete chemical entities. A “composition” or a “compound” shall also be taken to encompass other composition of matter such as cells e.g., ECs and other cells types such as that of therapeutic/prophylactic benefit.

As used herein, the terms “prevention” and “treatment” shall not be taken to require an absolute i.e., 100% abrogation of impaired vascular complication and/or atherosclerosis, or an absolute i.e., 100% prevention of the development of vascular complication and/or atherosclerosis in a subject having risk factors thereof, and it is sufficient that there is significant reduction in the adverse vascular effect(s) using the method of the present invention compared to the absence of prophylaxis or therapy in accordance with the present invention.

Similarly, the term “alleviating” or “alleviate” as used throughout this specification shall not be taken to require abrogation of impaired vascular function or vascular complication and/or atherosclerosis in a subject that is more than a significant effect compared to the absence of treatment in accordance with the present invention.

Similarly, the terms “modulate”, “promote”, “enhance”, “agonize”, “induce”, “activate”, “antagonize” “repress”, “delay”, and “inhibit” as used throughout this specification shall not be taken to require any particular quantitative change, merely a modified level and/or activity and/or expression, or modified timing thereof, that is significant compared to the absence of the composition or treatment in accordance with the present invention.

As used herein, the term “administer” shall be taken to mean that a composition that modulates monocytes differentiation into non-haematopoietic cells is provided or recommended to a subject in need thereof e.g., in a single or repeated or multiple dosage by any administration route. In one example, the composition is administered by oral means e.g., as a tablet, capsule, liquid formulation. In another example, the composition is administered by inhalation or apsiration e.g., as a powder via the respiratory system of the subject including the nasal passage, buccal cavity, throat or eosophagus or lung. In another example, the composition is administered to the circulatory system of a subject by injection e.g., intramuscularly, subcutaneously, intravenously, intraperitoneally.

A “subject” to which the present invention can be applied is a human or other mammalian subject capable of developing one or more vascular complications and/or atherosclerosis, including e.g., a domesticated animal such as a domestic pet or commercially-valuable animal. The prophylactic or therapeutic treatment of a dog, cat or horse is clearly encompassed by the present invention.

As used herein, the term “subject in need thereof” shall be taken to mean a subject that has developed or suffers from or one or more vascular complications and/or atherosclerosis, and/or is predisposed by virtue of having one or more risk factors to suffering from one or more vascular complications and/or atherosclerosis. In one example, the subject has already suffered from one or more vascular complications and/or atherosclerosis and/or has aberrant endothelial maintenance and/or repair processes, aberrant endothelium morphology and/or endothelial cell proliferation potential and/or activity. In another example, the subject has not yet suffered apparent impairment of vascular function or any apparent vascular complication and/or atherosclerosis, however has one or more risk factors for a vascular complication and/or atherosclerosis. In another example, the subject is a subject in need of vascular maintenance and/or repair or enhanced vascular maintenance and/or repair.

The present invention clearly contemplates repeated administration of a composition as described according to any example hereof in the therapy or prophylaxis of vascular complications and/or atherosclerosis. For example, repeated injection and/or inhalation of a composition of the present invention may be required to enhance or promote monocytes differentiation into non-haematopoietic cells in the subject e.g., in the circulatory system, for a prolonged period of time. Repeated administration may be timed so as to ensure a sufficiently high concentration of the bioactive composition in plasma of the subject and/or at the site of action in the treatment regimen. For example, second and/or subsequent doses may be administered at a time when serum concentration provided by one or more previous doses fall(s) below a desired level at which it is active or provides sufficient benefit to the patient. Such booster doses are clearly contemplated in the prophylaxis and/or therapy of one or more vascular complications and/or atherosclerosis and/or to promote vascular maintenance and/or repair and/or to enhance vascular maintenance and/or repair according to the present invention.

Substrate monocytes or substrate ECs of human origin, or potentially cells of porcine origin, are preferred for medical applications. More preferably the cells are derived from a tissue of a subject to whom a downstream product thereof is to be administered i.e., they are autologous.

For veterinary or animal improvement purposes, the cells may be derived from any animal species in the substrate monocytes and the substrate ECs would be compatible when used in the assay. Cells from any commercially-important animal species are contemplated herein e.g., pigs, cattle, horses, sheep, goats, dogs, cats, etc. As with human applications, it is preferred to use autologous cells for such applications to maximize compatibility between substrate monocytes and substrate ECs and/or to minimize rejection if a down stream product thereof is administered to the animal.

### 3. General

Unless the context requires otherwise or specifically stated to the contrary, integers, steps, or elements of the invention recited herein as singular integers, steps or elements clearly encompass both singular and plural forms of the recited integers, steps or elements.

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, developmental biology, mammalian cell culture, recombinant DNA technology, histochemistry and immunohistochemistry and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Methods In Enzymology (S. Colowick and N. Kaplan, eds., Academic Press, Inc.), whole of series;

### BRIEF DESCRIPTION OF THE DRAWINGS

FIG. 1 is a graphical representation showing flow cytometry density plot analysis of Human Umbilical Vein ECs (HUVECs) and Peripheral Blood Mononuclear Cells Culture (PBMCs) in co-culture. Two distinct cell populations based on HLA-A2 expression were clearly observed after two (2) hours in PBMC/HUVEC co-culture. The HUVEC cell population was HLA-A2 negative and constituted 81.4% of total cells, and the PBMC-derived cell population was HLA-A2 positive and constituted 18.6% of total cells.

FIG. 2 is a graphical representation showing flow cytometry density plot analysis of HLA-A2 positive PBMC derived monocytes which trans-differentiate into ECs and/or EC-like cells during co-culture with HUVECs over a one, two and three-day period. Panel A, shows expression of cell surface antigen CD14 as a marker of monocytes after 2 hours of PBMCs/HUVECs co-culture (day 0) and after 24 hours of PBMCs/HUVECs co-culture (day 1). Panel B, shows expression of CD11b after 2 hours of PBMCs/HUVECs co-culture (day 0) and after 72 hours of PBMCs/HUVECs co-culture (day 3). CD14 was down-regulated in HLA-A2 positive PMBC-derived cells following 1-day co-culture of PBMCs with HUVECs and CD11b was down-regulated in HLA-A2 positive PBMC-derived cells following 3-days co-culture of PBMCs with HUVECs.

FIG. 3 is a graphical representation showing flow cytometry density plot analysis of HLA-A2 positive PBMC derived monocytes which trans-differentiate into ECs and/or EC-like cells during co-culture with HUVECs following a six-day period. Panel A, shows expression of CD31 after six days of PBMCs/HUVECs co-culture (day 6). Panel B, shows expression of ICAM-1 after six days of PBMCs/HUVECs co-culture (day 6). CD31 and ICAM-1 were expressed by both HUVECs and by the HLA-A2 positive (PBMC-derived cells) throughout the 6 days co-culture period.

FIG. 4 is a graphical representation showing flow cytometry density plot analysis of HLA-A2 positive PBMC derived monocytes after 2 hours co-culture with HUVECs. PBMCs freshly isolated from HLA-A2 positive donors were incubated with HLA-A2 negative HUVECs at a dose of 1×10⁶ cells per well for 2 hours, and non-adherent PBMCs were removed by washing. The co-cultured layers were then dissociated and analyzed with flow cytometry. Panel A, shows expression of CD34 after 2 hours. Panel B, shows expression of KDR 2 hours. The PBMCs that adhere to the HUVECs were CD34 negative and KDR negative.

FIG. 5 is a graphical representation showing flow cytometry density plot analysis of HLA-A2 positive monocytes which trans-differentiate into ECs and/or EC-like cells (M-ELCs) during co-culture with HUVECs over a two-day period. Panel A, shows expression of CD105 as a marker of ECs and/or EC-like cells during co-culture of HUVEC and PBMCs derived monocytes after 2 hours (day 0) and at day 2. Panel B, shows expression of CD144 as a marker of ECs and/or EC-like cells during co-culture of HUVEC and PBMCs derived monocytes at after 2 hours (day 0) and at day 2. HLA-A2 positive monocyte-derived EC like cells (M-ELCs) acquired the CD105 and CD144 markers within 2 days following co-culture of the monocytes with HUVECs, indicating that they are ECs and/or EC-like cells.

FIG. 6 is a graphical representation showing flow cytometry density plot analysis of HLA-A2 positive monocytes which trans-differentiate into monocyte derived ECs and/or EC-like cells (M-ELCs) during co-culture with HUVECs following a five-day period. This figure shows expression of VCAM-1 during co-culture of HUVEC and PBMCs derived monocytes after 5 days of co-culture without TNFα stimulation (Panel A) and after 5 days of co-culture with TNFα stimulation (Panel B). Approximately half of the HLA-A2 positive monocyte derived EC like cells expressed VCAM-1 by day 5 after TNFα stimulation.

FIG. 7 is a graphical representation showing flow cytometry density plot analysis of HLA-A2 positive monocytes which trans-differentiate into monocyte derived ECs and/or EC-like cells (M-ELCs) during co-culture with HUVECs following 2 hours (day 0) and 24 hours co-culture (day 1). HLA-A23 positive PBMCs were incubated at a dose of 1×10⁶ cells per well with HLA-A2 negative HUVECs, after which point the non-adherent cells were washed off and the cells layers with dual colour flow cytometry. Panel A, shown CD36 expression representative of 2 independent experiments following 2 hours co-culture (day 0) demonstrating that the majority of the endothelial-adherent PBMCs expressed CD36 at day 0. Panel B, shows CD36 expression representative of 4 independent experiments following 24 hours of co-culture demonstrating that only 10% of the PBMC-derived cells were CD36 positive after 1 day in co-culture.

FIG. 8 is a graphical representation showing percentage (%) of HLA-A2 positive cells over time (at day 1 and day 3) during co-culture with HUVEC(HLA-A2 negative) cells. The proportion of monocyte-derived ECs and/or EC-like cells remain stable under culture conditions even after three days in culture. Groups were compared using Student's t-test (P, not significant).

FIG. 9 is a graphical representation showing quantification (%) of HLA-A2 positive monocytes derived EC-like cells with varying PBMC seeding density in a co-culture of PBMCs with HUVECs (HLA-A2 negative). PBMCs seeding densities of 0.25×10⁶ cells per well (low density), 0.5×10⁶ cells per well (intermediate density) and 1×10⁶ cells per well (high density) conditions were investigated. Groups were compared using Student's t-test. The percentage of HLA-A2 positive monocytes derived EC-like cells in the co-culture was dose dependent on the PBMCs seeding density with P<0.01 for differences between the low, intermediate and high density results.

FIG. 10 is a representation showing quantification of HLA-A2 positive monocytes derived EC-like cells with varying EC (HUVEC) seeding density in a co-culture of PBMCs with HUVECs (HLA-A2 negative). HUVECs were cultured on 6-wells cell culture plates (9.6 cm² plating surface area/well) at densities of 0.1×10⁶ cells per well (low density), 0.2×10⁶ cells per well (intermediate density) and 0.3×10⁶ cells per well (high density). The PBMC seeding density was 1×10⁶ cells per well. Panel A, shows total cell counts after 24 hours of co-culture. Panel B, shows proportions of HLA-A2 positive monocytes derived EC-like cells in the co-culture after 24 hours. The proportion of monocyte-derived ECs and/or EC-like cells in the co-culture was not affected by varying the EC substrate cell density.

FIG. 11 is a graphical representation showing the apolipoprotein A-I (ApoA-I) enhanced endothelial incorporation of monocyte-derived ECs and/or EC-like cells. ApoA-I was added to a co-culture of HLA-A2 positive monocytes and HLA-A2 negative HUVECs at concentrations of 0 mg/ml, 0.25 mg/ml, 0.5 mg/ml and 2.0 mg/ml. Percentages of adherent HLA-A2 positive cells of the total cells in the endothelial cell layer were analyzed on day 0 and day 3 following co-culture. Day 3 results are shown. ApoA-I increased endothelial incorporation of HLA-A2 positive monocyte-derived ECs and/or EC-like cells into the endothelial layer, in a dose responsive fashion.

FIG. 12 is a graphical representation showing the effects of LDL on the proportion (% of total cells) of adherent monocyte-derived HLA-A2 positive cells incorporated into the endothelial layer. Human LDL isolated from fresh human plasma was added to a test co-culture of HLA-A2 positive monocytes and HLA-A2 negative HUVECs at a protein concentration of 0.5 mg/ml, but not to a control co-culture of monocytes and HUVECs. Percentage of adherent HLA-A2 positive cells of the total cell count in the endothelial cell layer was analyzed on day 1 and day 2 following co-culture. The mean and standard deviation of the results obtained from both day 1 and day 2 are shown. Human LDL significantly reduced the incorporation of HLA-A2 positive monocyte-derived ECs and/or EC-like cells into the endothelial layer during co-culture with HUVEC.

### DETAILED DESCRIPTION OF THE PREFERRED EMBODIMENTS

### Substrate Cells

In one example, the present invention utilizes substrate monocytes and substrate ECs to produce ECs and/or EC-like cells from the substrate monocytes.

Any differentiated monocytes may be employed, e.g., those obtained and/or derived from an animal, including terminally differentiated monocytes. For example, the substrate monocytes may be primary cells, a cell strain or a cell line, and may be commercially available.

Any differentiated ECs may be used as substrate ECs e.g., obtained and/or derived from an animal, including terminally differentiated ECs. For example, the substrate ECs may be primary cells, a cell strain or a cell line, and may be commercially available.

By “differentiated” it is meant a cell that expresses defined specialized properties that are characteristic of that cell type and capable of being passed onto daughter cells on cell division. A differentiated cell need not be an actively proliferating cell. Methods for determining the expressed specialized properties will be apparent to the skilled artisan and/or described herein.

The substrate monocytes and/or the substrate ECs may be readily available in substantial quantities. Alternatively, or in addition, the substrate monocytes and/or the substrate ECs may be expanded in cell culture prior to use in the method of the invention. Methods for expanding cells in culture will be apparent to the skilled artisan and/or described herein.

Alternatively or in addition, the substrate monocytes and/or substrate ECs may be obtained or derived from any animal and preferably from a mature adult animal. The type of animal preferably includes but is not limited to humans, and includes any animal species such as: other primate such as ape, chimpanzee, gorilla, monkey, or orangutan, horse, cow, goat, sheep, pig, dog, cat, bird, fish, rabbit, rodent, such as a mouse or rat.

The substrate monocytes and/or substrate ECs may be purchased from any commercial source including PromoCell® (Banskia Scientific Company, QLD, Australia).

### Substrate ECs

The substrate ECs for use in the method of the invention may be derived from any source of mature endothelial cell known in the art. In a preferred non-limiting embodiment the types of ECs that are used as substrate in the method according to this invention are human umbilical vein ECs (HUVEC) preferably isolated from human umbilical veins using collagenase digestion. In a further preferred embodiment human coronary artery ECs (HACECs) may be used as substrate ECs.

As a further non-limiting example, the ECs substrate may be derived from freshly isolated dermal ECs and/or adipose ECs and/or microvascular ECs and/or arterial ECs and/or choroid plexus endothelial cells and/or dermal microvascular ECs and/or lymphatic ECs e.g., lymph nodes ECs and/or intestinal microvascular ECs and/or Cardiac ECs and/or aortic ECs and/or umbilical vein ECs and/or umbilical artery endothelial. Alternatively, commercially available ECs that can be used in the method of the invention, including but not limited to HBMEC (Human Brain Microvascular ECs), HCPEC (Human Choroid Plexus ECs), HDMEC (Human Dermal Microvascular ECs), HDLEC (Human Dermal Lymphatic ECs), HLEC-ln (Human Lymphatic ECs-lymph nodes), HIMEC (Human Intestinal Microvascular ECs), HPMEC (Human Pulmonary Microvascular ECs), HPAEC (Human Pulmonary Artery ECs), HRGEC (Human Renal Glomerular ECs), HHSEC (Human Hepatic Sinusoidal ECs), HCMEC (Human Cardiac Microvascular ECs), HAEC (Human Aortic ECs), HREC (Human Retinal ECs), HUVEC (Human Umbilical Vein ECs), HUAEC (Human Umbilical Artery ECs) (Sciencecell Research Laboratories, San Diego, Calif.).

In another example of the present invention, non-human ECs may also be used. In one non-limiting example, C57B/6CJ mouse microvascular ECs which are available commercially (American Type Culture Collection) may be grown to confluence in endothelial cell basal medium, supplemented with 10% FCS, penicillin, streptomycin and endothelial cell growth supplement/heparin (Clonetics). Such cells may be passaged one or more times e.g., twice, and used as substrate ECs in the method of the present invention.

### Substrate Monocytes

In a preferred embodiment, peripheral blood mononuclear cells (PBMCs) provide a readily available source of substrate monocytes for use in the method of the invention. Various methods are also available for separating out and/or enriching cultures of PBMCs, including indirect binding of PBMCs to specifically-coated surfaces. In another example, PBMCs may be obtained by density gradient separation. PBMCs may also be obtained by density gradient separation of ethylenediaminetetra-acetic acid (EDTA) and/or heparin and/or ACD-anti-coagulated venous blood drawn from subjects. In one example, the PBMCs can be obtained from venous blood by gradient centrifugation, e.g. using Lymphoprep (Lymphoprep™) in a hypertonic medium, e.g., medium containing 0.9% NaCl. Suitable techniques of gradient separation or PBMCs on Lymphoprep are known in the art, see, for example, Májský A. Cas Lek Cesk. 1989 Dec. 8; 128(50):1591-3; Boyum A. Scand J Clin. Lab. Invest. 21, Suppl. 97. 1968; Harris, R et al. Lancet. 1969, 327:7615. In another example, PBMCs can be obtained by Ficoll-Hypaque density gradient separation of ethylenediaminetetraacetic acid (EDTA)-anti-coagulated venous blood (Pharmacia, Piscataway, N.J.).

PBMCs may be used immediately following density gradient separation, by seeding onto cultured ECs. Alternatively, PBMCs may be stored in suitable medium to conserve cell viability and function and frozen. For example cells are stored in FBS containing 10% DMSO (Sigma Chemical Co., St. Louis, Mo.) and stored under liquid nitrogen. In a further example, after separation and prior to seeding, the PBMCs are washed with a suitable salt solution, such as Seligman's balanced salt solution (SBSS), which is obtainable from Gibco, Grand Island, N.Y.), and then resuspended in a suitable medium, such as M199 or RPMI 1640 medium, which are obtainable from M. A. Bioproducts, Walkerville, Md., supplemented with 1 mM L-glutamine solution, 1% penicillin streptomycin solution, optionally 1% antibiotic-antimycotic solution, 20 mM HEPES (Gibco, Grand Island, N.Y.) and 10% heat inactivated human serum (percentages are v/v unless otherwise specified). Other suitable media are well known, see, for example, Rosenberg, et al., The New England Journal of Medicine 313, 1485, 1486 (1985).

Alternative methods of separating and/or enriching cultures of monocytes include both positive and negative selection procedures. For positive selection, PBMC populations are prepared from whole blood, e.g., by utilization of antigenic determinants on the cell surface, their reactivity with antibodies which fluoresce under known circumstances, and e.g., by utilization of principles of flow cytometry. In one example, PBMCs, such as monocytes may be isolated by affinity-based separation techniques, for example directed at the presence of the CD4 co-receptor antigen. These affinity-based techniques fluorescence-activated cell sorting (FACS), cell adhesion, magnetic bead separation and like methods. (See, e.g., Scher and Mage, in Fundamental Immunology, W. E. Paul, ed., pp. 767-780, River Press, NY (1984)) Affinity methods may utilize for example anti-CD4 co-receptor antibodies as the source of affinity reagent. Alternatively, the natural ligand, or ligand analogs, for example of CD4 receptor may be used as the affinity reagent. Various anti-T cell and anti-CD4 monoclonal antibodies for use in these methods are generally available from a variety of commercial sources, including the American Type Culture Collection (Rockville, Md.) and Pharmingen (San Diego, Calif.).

Negative selection procedures are utilized to effect the removal of non-monocytes from the PBMC population, based on their T cell antigen designation. This technique results in the enrichment of cells expressing monocytes antigens. Depending upon the antigen designation of the T cells to be depleted, different antibodies may be appropriate. For example, monoclonal antibodies OKT4 (anti-CD4, ATCC No. CRL 8002) OKT 5 (ATCC Nos. CRL 8013 and 8016), OKT 8 (anti-CD8, ATCC No. CRL 8014), and OKT 9 (ATCC No. CRL 8021) are identified in the ATCC Catalogue of Cell Lines and Hybridomas (ATCC, Rockville, Md.) as being reactive with human T lymphocytes, human T cell subsets, and activated T cells, respectively. Various other antibodies are also available for identifying and isolating T cell species of the PBMC populations.

Contacting Substrate Monocytes with Substrate ECs

The present invention clearly encompasses the use of any time and/or any condition parameters sufficient for non-haematopoietic cells to be produced from the substrate monocytes. It is well within the ken of a skilled addressee to determine such parameters without undue experimentation. For example, the time of incubation of substrate monocytes with substrate ECs may be determined by trypsinization of cultures at different times and measuring phenotypic expression of cell surface markers of non-haematopoietic cells, as determined such as for example by immunoassay and/or flow cytometry.

For example, the time sufficient for non-haematopoietic to be produced is less than 4 days, more preferably less than 3 days, more preferably less than 2 days, more preferably within 24 hours from contact of the substrate monocytes with the substrate ECs.

It will be apparent that the trans-differentiation of substrate monocytes to EC-like cells by the method of the present invention is advantageously much more rapid and advantageously involves a much greater proportion of substrate monocytes than previously achieved in the art. The rapidity of the monocyte differentiation into EC or EC-like cells using the method of the invention, which may be apparent within 24 to 48 hours contrasts with prior methods requiring about 7 to 14 days for monocytes to acquire endothelial antigens.

### Assessment of Cell Phenotype

In one example, the substrate monocytes and the produced non-haematopoietic cells express the same alleles of a detectable marker and wherein the substrate ECs may express one or more different alleles of a detectable marker to thereby distinguish the substrate ECs from the substrate monocytes and from the produced non-haematopoietic cells e.g., ECs and/or EC-like cells.

For example, the detectable marker may be a marker that is capable of being expressed by both substrate monocytes and the non-haematopoietic cells produced from the substrate monocytes. Preferably, the marker is a cell surface antigen capable of being expressed by both substrate monocytes and the non-haematopoietic cells e.g., ECs and/or EC-like cells produced from the substrate monocytes.

By determining expression of one or more different alleles of a detectable marker in the substrate monocytes, and/or the produced non-haematopoietic cells e.g., ECs and/or EC-like cells, and/or the substrate ECs, the expression of different alleles of the detectable marker in the substrate ECs to the alleles of the detectable marker expressed by the substrate monocytes and produced non-haematopoietic cells may indicate non-haematopoietic cell production e.g., EC production and/or EC-like cell production from the monocytes.

The detectable marker need not be functional or associated with a particular disease or condition. In one example, the detectable marker is a human leukocyte antigen (HLA), e.g., HLA type A2 (HLA-A2). For example, if the substrate ECs do not express HLA-A2 i.e., are HLA-A2 negative, and the substrate monocytes are HLA-A2 positive, then non-haematopoietic cell production from the substrate monocytes is assessed by monitoring expression of HLA-A2 in combination with one or more markers of the non-haematopoietic cells e.g., ECs and/or EC-like cells. According to this preferred example, expression of the HLA-A2 antigen by the non-haematopoietic cells is indicative of non-haematopoietic cell production from the substrate monocytes.

Alternatively, the substrate ECs may express a marker not present on the substrate monocytes. For example, substrate ECs may express HLA-A2 i.e., may be HLA-A2 positive, and the substrate monocytes and non-haematopoietic cells produced from the substrate monocytes may not express HLA-A2 i.e., may be HLA-A2 negative. According to this example, lack of expression of the marker by the substrate monocytes and by the non-haematopoietic cells e.g., ECs and/or EC-like cells is indicative of non-haematopoietic cell production from the substrate monocytes.

As a further non-limiting example, the detectable marker expressed on one of the substrate monocytes or substrate ECs, but not both cell types may be selected from HLA-A, HLA-B, HLA-C, HLA-DPA1, HLA-DPB1, HLA-DQA1, HLA-DQB1, HLA-DRA, and HLA-DRB1, or a Y chromosome antigen, such as for example the H-Y antigen, or any one or more of detectable markers that allow mistyping of substrate endothelial cell and/or substrate monocytes and/or the non-haematopoietic cells e.g., ECs and/or EC-like cells produced from the substrate monocytes.

The term “allele” as used herein shall be understood in its broadest context to refer to any one of the different forms of a gene or DNA sequence at a single locus i.e., chromosomal location including a coding sequence, non-coding sequence or regulatory sequence, which codes for the detectable marker, and which results in a detectable polymorphism and/or quantitative trait variation associated with the expression of the detectable marker by a cell. The term “allele” also encompasses a null-allele resulting in lack of expression of the detectable marker by a cell.

In one example, the method may comprise determining expression of the detectable marker by the substrate monocytes and/or the substrate ECs. In another example, the method may comprise determining expression of the detectable marker by the substrate ECs and/or the produced non-haematopoietic cells e.g., ECs and/or EC-like cells. In yet another example, the method may comprise determining expression of the detectable marker by the substrate monocytes and/or the substrate ECs and in the produced non-haematopoietic cells e.g., ECs and/or EC-like cells.

In one example, the substrate monocytes and/or produced non-haematopoietic cells e.g., ECs and/or EC-like cells are determined by the presence of absence of one or more surface cell-type specific markers.

In one preferred example, substrate monocytes are determined by the presence of one or more monocyte-specific markers including but not limited to CD14 and/or CD11b. In one example, the substrate monocytes are determined by the presence of the class B scavenger receptor CD36. Alternatively or in addition thereto, substrate monocytes are determined by the absence of one or more cell surface markers including but not limited to CD34, KDR (also known as VEGF-R2), Tie-2, CD144 and/or CD105, but not determined by the presence or absence of CD31 marker. Alternatively or in addition thereto, substrate monocytes are determined by the absence of one or more progenitor cell-surface specific marker, such as for example CD133. Alternatively or in addition thereto, substrate monocytes are determined by the absence of one or more macrophage or dendritic cell surface marker specific, such as for example CD115.

In one preferred example, substrate ECs and/or monocyte-derived non-haematopoietic cells e.g., ECs or EC-like cells produced by the method according to the invention, are determined by the presence of expression of one or more of the endothelial cell-specific markers including but not limited to CD105, CD144, CD146 and/or VCAM-1. Alternatively or in addition thereto, monocyte-derived non-haematopoietic cells produced by the method according to the invention, are determined by the absence of expression of one or more cell surface markers such as for example CD14 and/or CD11b and/or CD115 and/or CD36. Alternatively or in addition thereto, the monocyte-derived non-haematopoietic cells produced by the method according to the invention are determined by the absence of expression of the endothelial progenitor cell (EPC)-surface marker, such as for example CD133.

### Quantification of Non-Haematopoietic Cell Product

In one example, the present invention contemplates quantifying production of non-haematopoietic cells e.g., ECs and/or EC-like cells, from substrate monocytes which comprises determining the ratio or percentage or number of substrate monocytes that differentiate into non-haematopoietic cells such as for example, ECs and/or EC-like cells.

Such quantification may employ any one of a number of commercially available antibodies, immunofluorescence or flow cytometry techniques. For example, cell cultures are liberated or detached by any method known in the art or as described infra, contacted with a detectably-labelled antibody that binds selectively to a marker on a cell type being detected e.g., substrate monocyte, substrate EC or non-haematopoietic cell product, and the amount of detectable label bound to the cells is determined. In one format of this example, cultures are liberated by trypsin/EDTA digest, washed and resuspended in 200 μl of a primary antibody comprising a mouse IgG monoclonal antibody or rabbit IgG that binds to the marker (e.g., DAKO, Santa Cruz, Pharmingen, or Sigma), then washed and contacted with a goat anti-mouse IgM μ-chain specific-FITC and either goat anti-mouse IgG γ-specific-PE or anti-rabbit Ig-specific-PE (Southern Biotechnology Associates), washed and fixed in FAX FIX (PBS supplemented with 1% (v/v), 2% (w/v) D-glucose, 0.01% sodium azide). Flow cytometric analysis is performed using a FACSCalibur flow cytometer and the CellQuest software program (Becton Dickinson Immunocytometry Systems, San Jose, Calif.). Data analysis is performed using CellQuest and the Modfit LT V2.0 software program (Verity Software House, Topsham, Me.).

It will be apparent to the skilled addressee that in contrast to the present invention, other methods known in the art that require culturing monocytes on matrix protein-coated culture surfaces in the absence of endothelial cells and/or which use circulating EPCs are not quantitative. In those known methods, the proportions of the substrate monocytes that adhere to the culture surface are not known and circulating EPCs comprise less than 1% of PBMCs, as determined by quantifying blood cells that express antigens such as CD34, KDR and CD133. In contrast, the method of the present invention permits quantitation of the number of EC-like cells produced from substrate monocytes.

### Detachment of Adherent Cells in Culture

In one example, the quantification of non-haematopoietic cell product such as for example, ECs and/or EC-like cells, includes detachment of adherent cells in culture. The means by which adherent cells in culture are detached from each other and/or from the culture vessel may be varied. The present invention clearly encompasses the use of any means by which adherent cells in culture are detached from each other and/or from the culture vessel as described by Ivaska and Heino, Cell. Mol. Life. Sci. 57:16-24 (2000) and references described therein.

In a preferred embodiment, adherent cultures are detached from tissue culture plates by incubation of the adherent cells in trypsin for a time and under conditions sufficient for detachment to occur e.g., as described in the Examples.

Trypsin may be purchased from a variety of commercial sources in stock concentrations up to about 2.5% (w/v) trypsin, such as, for example, from GIBCO (Invitrogen). The final trypsin concentration used to achieve detachment when using such a solution is preferably about 0.01% (w/v) to about 0.25% (w/v) trypsin, including about 0.05% (w/v), or about 0.10% (w/v), or about 0.11% (w/v), or about 0.12% (w/v), or about 0.13% (w/v), or about 0.14% (w/v), or about 0.15% (w/v), or about 0.16% (w/v), or about 0.17% (v/v) or about 0.18% (w/v) or about 0.19% (w/v) or about 0.2% (w/v) or about 0.25% (w/v).

It will be apparent to the skilled artisan that the time of incubation in trypsin solution may vary according to cell type, and it is well within the ken of a skilled addressee to determine such parameters without undue experimentation. For example, the time of incubation in trypsin solution is sufficient for the cells to lift from the plates and/or preferably, to detach from each other as determined by the degree of cell clumping or aggregation.

It will also be apparent to the skilled artisan that the temperature for the incubation in trypsin solution is preferably between about 15° C. and about 37° C., or preferably room temperature, or more preferably 37° C. By “room temperature” is meant ambient temperature e.g., between about 18° C. and about 25° C.

Other suitable methods for achieving detachment of cells from each other and/or from the culture vessel include, but are not limited to, cold shock; treatments to release integrin receptors from the extracellular matrix, which comprises fibronectin, vitronectin, and one or more collagens; activation of degradation of matrix molecules including, but not limited to fibronectin, collagens, proteoglycans, and thrombospondin; inducing or enhancing the secretion of proteases, such as, but not limited to collagenase, stromelysin, matrix-metalloproteinases (MMPs; a class of structurally related zinc-dependent endopeptidases that collectively degrade extracellular matrix components) or plasminogen activator; and decreasing or repressing the expression of protease inhibitors, plasminogen activator inhibitor (PAI-1) or tissue inhibitors of metalloproteinases (TIMPs). Such methods are described without limitation for example, by Ivaska and Heino, Cell. Mol. Life. Sci. 57:16-24 (2000), Nagase et al., Cardovasc. Res. 69(3):562-73 (2006) or a reference cited therein. Preferred means for inducing or enhancing MMP expression include induction by addition of growth factor or cytokine to the culture medium.

Preferred cold shock means comprise incubating the cells in ice-cold phosphate buffered saline (PBS) or other isotonic buffer for a time and under conditions sufficient for detachment to occur. Preferred conditions include cold shock for about 10 minutes or until the cells lift from the plates and/or detach from each other as determined by the degree of cell aggregation.

Integrin receptors can be released from the extracellular matrix by incubating the cells with a synthetic peptide containing the Arg-Gly-Asp sequence that competes for binding to the integrin receptors such as described, for example, by Haymen et al., Journal Cell Biol, 100:1948-1954 (1985). Alternatively, or in addition, integrin receptors are released from extracellular matrix by incubating cells in a Ca²⁺-free and Mg⁺-free solution comprising EDTA (e.g., Ca²⁺-free and Mg²⁺-free PBS comprising EDTA, or other Ca²⁺-free and Mg⁺-free isotonic buffer) essentially as described by Sambrook, Fritsch & Maniatis, Molecular Cloning: A Laboratory Manual, Cold Spring Harbor Laboratories, New York, Second Edition (1989), whole of Vols I, II, and III; Animal Cell Culture: Practical Approach, Third Edition (John R. W. Masters, ed., 2000), ISBN 0199637970, whole of text; Methods In Enzymology (S. Colowick and N. Kaplan, eds., Academic Press, Inc.), whole of series.

### Assay Platforms

The production of non-haematopoietic cells such as ECs and/or EC-like cells from substrate monocytes and/or the incorporation of the monocyte-derived non-haematopoietic cells into the endothelium is carried out in the presence of a sample, to thereby determine the ability of the sample or a fraction or molecule in the sample to modulate non-haematopoietic cell production from substrate monocytes or incorporation of the non-haematopoietic cells into endothelium. The sample may be a biological sample or composition of matter e.g., peptidyl compound, antibody, nucleic acid or small molecule.

In one example, the sample may lack monocytes or be monocyte-depleted, wherein the method may also comprise contacting exogenous substrate monocytes with substrate ECs in the presence and/or absence of the sample for a time and under conditions sufficient for non-haematopoietic cells to be produced from the substrate monocytes. In accordance with this example, an inherent ability of factors in the sample influencing production of non-haematopoietic cells from exogenous substrate monocytes in the presence of substrate ECs is determined. The sample may be from a subject having one or more vascular complications and/or atherosclerosis and/or is at a risk of developing one or more vascular complications and/or atherosclerosis. Alternatively, the sample may comprise one or more compositions of matter to be screened for their ability to modulate non-haematopoietic cell production or incorporation into endothelium e.g., vascular endothelium.

In this context, the term “exogenous substrate monocytes” includes but is not limited to substrate monocytes which are derived, isolated or obtained from a source other than the source from which the sample has been derived, isolated or obtained.

In another example, the production of non-haematopoietic cells such as ECs and/or EC-like cells from substrate monocytes and/or the incorporation of the monocyte-derived non-haematopoietic cells into the endothelium is carried out in the presence of a sample that provides the substrate monocytes. That is, the substrate monocytes are endogenous substrate monocytes. In accordance with this example, the inherent ability of a patient's monocyte population to produce non-haematopoietic cells e.g., ECs and/or EC-like cells is determined. The sample may be from a subject having one or more vascular complications and/or atherosclerosis and/or is at a risk of developing one or more vascular complications and/or atherosclerosis.

In another example, the production of non-haematopoietic cells such as ECs and/or EC-like cells from substrate monocytes and/or the incorporation of monocyte-derived non-haematopoietic cells into the endothelium is carried out in the presence of a sample that provides substrate ECs or one or more factors normally produced by substrate ECs. That is, the substrate ECs are endogenous substrate ECs. In accordance with this example, the inherent ability of a patient's sample to substitute a requirement for substrate ECs is determined. The sample may be from a subject having one or more vascular complications and/or atherosclerosis and/or is at a risk of developing one or more vascular complications and/or atherosclerosis.

In yet another example, the production of non-haematopoietic cells in the presence of a patient sample that is either monocyte-depleted or comprises substrate monocytes or comprises substrate ECs is determined in the presence and/or absence of an agonist, a partial agonist, an antagonist or inverse agonist of monocyte differentiation into non-haematopoietic cells and/or incorporation of non-haematopoietic cells into endothelium. Preferably, the production of non-haematopoietic cells such as ECs or EC-like cells from substrate monocytes and/or the incorporation of the monocyte-derived non-haematopoietic cells into the endothelium is carried out in the presence of a composition comprising or consisting of an agonist or partial agonist of production of non-haematopoietic cell from monocytes. An agonist, partial agonist, inverse agonist or antagonist of monocyte differentiation into non-haematopoietic cells may be a composition of matter identified in a screening platform described herein.

The production of non-haematopoietic cells in the presence of an agonist, partial agonist, inverse agonist or antagonist of non-haematopoietic cell production may also be identified or isolated in the absence of substrate ECs. In a preferred form, an agonist or partial agonist promotes production of non-haematopoietic cells in the absence of substrate ECs i.e., by virtue of substituting for one or more factors produced and/or secreted by substrate ECs that otherwise permit or promote non-haematopoietic cell production from substrate monocytes. An agonist or partial agonist of monocyte differentiation into non-haematopoietic cells may therefor comprise an EC, a fraction of an EC or a factor produced by an EC, a serum-derived factor, or other factor that mimics the effect of an EC, mimics the effect of a fraction of an EC, mimics the effect of a factor produced by an EC, or mimics the effect of a serum-derived factor.

In a preferred format, the assay platforms are high-throughput assays e.g., conducted in multi-well microtiter plates or by high-throughput FACS to facilitate rapid screening of a plurality of candidate modularity compounds e.g., from a chemical compound library or peptide library or antibody library etc. It will be apparent to the skilled artisan that such high-throughput screens are generally but not necessarily, primary screens that may be validated in one or more cell-based assays, angiogenesis assays e.g., Matrigel, or in animal models of atherosclerosis. The assay platforms may also be performed for the purpose of validating primary screens, in which case high-throughput assays may not be required and, for example, they may be animal-based assays. Control assays wherein the candidate modulatory compound is omitted may also be performed.

### Cell-Based Assay Platforms

Cell-based assays may be employed for rapid determination of monocyte differentiation into non-haematopoietic cells such as ECs or EC-like cells and/or for rapid determination of the ability of a composition to modulate the production of non-haematopoietic cells such as ECs or EC-like cells from monocytes.

In one example of such assays, substrate ECs are passaged and cultured as endothelium in medium comprising e.g., serum such as a fetal calf serum or bovine calf serum or horse serum or human serum, preferably at a concentration of about 10% and preferably heat-inactivated serum. The cultures are preferably in cell culture plates, such as for example 6-well cell culture plates (Becton Dickinson Labware) or 96-well microtitre plates. Preferably, substrate ECs are cultured on plates for a number of passages, albeit preferably not more than two time, or three times or four times. Preferably, substrate ECs are passaged to a confluent or sub-confluent monolayer. More preferably, substrate ECs are grown to about 75% sub-confluence. In accordance with this example, substrate monocytes are seeded onto cultured ECs. The time and conditions or co-cultivation are generally sufficient for adherence of cells to occur. Preferably, the time and conditions or co-cultivation are generally sufficient for substrate monocytes to differentiate into non-haematopoietic cells e.g., ECs and/or EC-like cells. Preferably, differentiation occurs post-adherence. Preferably, the non-adhered cells are removed e.g., by washing. Preferably, adherent cell cultures are maintained in media supplemented with serum.

As will be apparent to the skilled artisan, the production of non-haematopoietic cells such as ECs or EC-like cells from substrate monocytes following contact of substrate monocytes with the substrate ECs and adherence of the non-haematopoietic cell to the endothelium in cell culture is indicative of differentiation of monocytes to non-haematopoietic cells such as ECs or EC-like cells and the incorporation of the monocyte-derived non-haematopoietic cells such as ECs or EC-like cells into the endothelium in vivo.

### In Vitro Angiogenesis Assay

The present invention encompasses the use of assay platforms comprising angiogenic assays, such as, for example, assays for determining proliferation, migration, and/or tubulogenesis of EC or EC-like cells, or the formation of vascular-like structures, including Matrigel assays. In one example, such angiogenic assays are utilized to determine an ability of non-haematopoietic cells such as ECs and/or EC-like cells derived from substrate monocytes to incorporate into endothelium.

It will be apparent to the skilled artisan that such assays are also useful in secondary screens of compounds that have been shown in high-throughput primary screens to modulate the production of non-haematopoietic cells from substrate monocytes. For example, such angiogenic assays are generally performed in the presence of a candidate modulatory compound identified e.g., by high-throughput screening, such as in a cell-based assay platform. Control assays wherein the candidate modulatory compound is omitted may also be performed.

A proliferation assay allows a determination of the number of cells that are growing in the absence or presence agents that modulate proliferation. Proliferation of non-haematopoietic cells such as ECs or EC-like cells may be determined by any means know in the art. Preferably, a cell proliferation assay is determined by EdU incorporation using Click-iT™ EdU Flow Cytometry Assay Kit according to the manufacture's protocol (Invitrogen). Alternatively, cell proliferation may be assayed using 5-bromo-2′-deoxyuridine (BrdU) incorporation into newly-synthesized cellular DNA which permits indirect detection of rapidly proliferating cells with fluorescently labeled anti-BrdU antibodies or nucleic acid stains, thereby facilitating the identification of cells that have progressed through the S-phase of the cell cycle during the BrdU labeling period.

Cell migration assays may be performed by any means know in the art. In one example, cell migration assays are performed in modified Boyden chamber comprising an endothelial transmigration layer in a 24 well plate format (Costar), wherein substrate ECs are cultured in the upper well of the chamber in media such as serum free M199 media supplemented with serum e.g., about 10% foetal bovine serum (FBS) or heat-inactivated human serum. Substrate monocytes are then seeded onto the cultured ECs and cultured with the monocytes. The time and conditions or co-cultivation are generally sufficient for adherence of cells to occur. Preferably, the time and conditions or co-cultivation are generally sufficient for substrate monocytes to differentiate into non-haematopoietic cells e.g., ECs and/or EC-like cells. Preferably, differentiation occurs post-adherence. Preferably, the non-adhered cells are removed e.g., by washing e.g., after about 3 hours. The adherent cell cultures may be maintained e.g., in media supplemented with serum such as about 10% FBS or heat-inactivated human serum. Preferably, non-haematopoietic cells e.g., ECs and/or EC-like cells labelled using a detectable label e.g., a radiolabel such as Cr⁵¹ or a fluorophore bound to an antibody or antibody conjugate that binds to an EC marker as described herein or to a lectin or lectin conjugate that selectively binds ECs, and their migration across the endothelial transmigration layer is determined. For example, the fluorophore may comprise FITC, wherein a membrane comprising the endothelial transmigration layer is removed and mounted in DAPI-containing aqueous VectaMount (Vector Laboratories) to thereby permit DAPI and FITC positive cells to be visualized by fluorescent microscopy (Olympus) and the number of ECs or EC-like cells migrating to the lower transwell side of the membrane calculated as the ratio of FITC-staining cells relative to DAPI-staining cells.

EC formation of vascular-like structures may also be assessed by the degree of invasiveness into Matrigel or some other extracellular matrix constituent. Matrigel assay provides an accepted in vitro model of angiogenesis that can be used to determine tubulogenesis of monocyte-derived ECs or EC-like cells following contact of the monocytes substrate with the endothelial substrate and incorporation of the monocyte-derived ECs or EC-like cells into endothelium. Invasiveness into Matrigel or some other extracellular matrix constituent can also be used as an assay to identify compounds that modulate production of monocyte-derived EC or EC-like cells and their incorporation into the endothelium.

In one example, EC formation of vascular-like structures on growth factor-reduced Matrigel (Becton Dickinson) is assayed in a method described by Weis et al., Art. Thromb. Vasc. Biol. 22, 1817-1823 (2002). Growth Factor Reduced Matrigel (BD) is added to each well of a 96-well plate and allowed to coat. Next, cells are added to each well and tubule formation observed over a period of 18 h. Using the Matrigel assay, contact of substrate monocytes with substrate ECs promotes new blood vessel formation. Preferably, outgrowth of new blood vessels from the endothelium occurs rapidly within about 4 days and more commonly preferably within about 48 hours after contact of substrate monocytes with substrate ECs.

Alternatively, the level of invasion EC or EC-like cells can be measured by using filters coated with Matrigel or some other extracellular matrix constituent. Penetration into the gel, or through to the distal side of the filter, is rated as invasiveness, and rated histologically by number of cells and distance moved, or by pre-labelling the cells with .sup.125I and counting the radioactivity on the distal side of the filter or bottom of the dish. See, e.g., Freshney (1994) Culture of Animal Cells: A Manual of Basic Technique (3d ed.) Wiley-Liss, herein incorporated by reference.

### Animal Models

A candidate compound for the treatment of one or more vascular complications and/or atherosclerosis capable of modulating production of monocyte-derived endothelial cell or EC-like cells that is determined using a cell based assay may be validated using an animal model. Such validation ensures that the compound only influences vascular maintenance and/or repair and/or progression of atherosclerosis, and is not, for example toxic to the animal. It will be apparent to the skilled artisan that animal models are generally employed by administering a candidate modulatory compound to animals and determining the effect of the compound or phenotype or the animals. The candidate modularity compound may be a compound identified e.g., by high-throughput primary screening. Control assays wherein the candidate modularity compound is omitted may also be employed.

A preferred model in which to validate a compound for the treatment of one or more vascular complications and/or atherosclerosis complications is apolipoprotein E-deficient (apoE^{-/-}) mice, such as for example C57BL/6JApoe<tmlUnc129> as an atherosclerosis prone mouse model. According to this example, control mice may include age and sex matched non-genetically modified C57BL/6J mice.

Non-mechanical injury to the endothelium of the mice may be induced by known methods in the art. Non-denuding endothelial injury may be induced in mice by intraperitoneal administration of lipopolysaccharide (LPS; Sigma-Aldrich) at a dose of 50 μg per animal. Untreated animal from the same batch of animals serve as controls.

Alternatively, injury to the endothelium vessel wall in mice may for example be photochemically-induced as described for example by Shinji Kikuchi et al., Arteriosclerosis, Thrombosis, and Vascular Biology. 18:1069-1078 (1998). In brief, mice are anaesthetized e.g., using sodium pentobarbital and a cannula is inserted into the jugular vein for rose Bengal injection. The right femoral artery is exposed, and the probe of a laser monitor is attached to the branch point of the deep femoral artery distal to the inguinal ligament for monitoring blood flow. Transillumination of the exposed segment with green light (wavelength 540 nm) is achieved by using a xenon lamp with a heat-absorbing filter and a green filter (Hamamatsu Photonics). A heat-absorbing filter may be used to prevent excessive generation of heat that can damage biological tissues. The irradiation is directed with a fiber-optic filament positioned 5 mm away from a segment of the intact femoral artery proximal to the flow probe. Irradiation at an intensity of 0.9 W/cm2 is optimal after baseline blood flow had stabilized. Rose Bengal is infused. The irradiated segment of the femoral artery is considered to be occluded by a platelet- and fibrin-rich thrombus when blood flow had completely stopped. The wound may be closed, and the animal allowed to returned to its cage after recovery from anaesthesia. Mice may be re-anesthetized at different time points, and spontaneous reflow may confirmed by monitoring the blood flow in the femoral artery. In photochemically induced thrombosis, spontaneous reflow after thrombotic occlusion of the femoral artery is affected by the duration of photo-irradiation, rose Bengal concentration, green light intensity, and the thickness of the vessel wall.

Preferably, a candidate compound identified in an assay platform of the invention promotes vascular maintenance and/or repair in a genetically modified animal subject, and more preferably, to levels similar to that observed in a wild-type animal. As will be apparent to the skilled artisan, a candidate compound that is validated in an animal model is then further validated in clinical trials.

### Assay Samples

The method of the present invention lends itself to the development of a wide range of assays for determining the effect of an exogenous composition e.g., a patient-derived sample or a compound in a screen, on the ability of monocytes to differentiate into ECs or endothelial-like cells and/or to determine an effect of the composition on one or more vascular maintenance and/or repair processes.

Such assays may be performed in vivo e.g., in animals such as accepted animal models of atherosclerosis and/or in vitro e.g., in high-throughput primary screens or other platforms described herein. It will be apparent from the disclosure herein that the nature of the assay sample will, to some extent, determine the assay platform utilized, and the application to which the read-out may be put. For example, assays for the identification or isolation of a composition having therapeutic efficacy in the treatment or prevention of atherosclerosis and/or for vascular maintenance and/or repair may employ substrate monocytes and a composition to be tested for agonism of the differentiation process in the absence of substrate ECs, become the ability of the composition to substitute for a requirement for substrate ECs is determined. In contrast, in screens for antagonists or inverse agonists of the differentiation process, both substrate monocytes and substrate ECs will generally be present, optionally with a known agonist of the process. Assays conducted using patient samples are generally of prognostic or diagnostic value, albeit not exclusively as such, will generally provide monocytes from the patient and carried out in the presence or absence of agonist, as for compound screens.

Assays of patient samples having diagnostic or prognostic value may also seek to determine the effect(s) of factors in the subject's circulation e.g., serum factors, on the ability of monocytes to engage in vascular maintenance and/or repair, in which case the subject's serum depleted of monocytes would be assayed for an ability to modulate differentiation of exogenous monocytes to ECs in the presence of exogenous ECs, optionally in the presence of a known agonist of the process.

In one example applicable to patient and/or compound screening, the sample being tested lacks monocytes or is monocyte-depleted. In one such example, the sample may comprise a composition capable of modulating production of non-haematopoietic cells from substrate monocytes (e.g., a modulatory composition as described infra). In another example, the sample is a biological sample isolated from a subject suspected of having one or more vascular complications and/or atherosclerosis and/or is at a risk of developing one or more vascular complications and/or atherosclerosis.

The term “biological sample” is intended to include but not be limited to, any tissue, cells and biological fluids (including, but not limited to blood, serum, plasma, lymph, cerebrospinal fluid, urine, fluid from body cavities such as the peritoneal space [ascites], pleural, pericardial, etc., cystic fluid, and exudates) isolated from a subject, including those obtained as surgical and biopsy samples, or from procedures such as washings of body cavities, as well as tissues, cells anal fluids present in vivo within the subject. Surgical and biopsy samples can be procured and samples stored under standard conditions to prevent degradation until the detection method can be performed. Such samples include preservation methods such as for example paraffination and/or freezing of fresh samples.

In another example applicable to patent screenings, an assay sample will comprise substrate monocytes. For example, the biological sample is a whole blood sample, fresh frozen whole blood sample, a blood plasma fraction, fresh frozen plasma fraction, and may comprise peripheral blood, umbilical cord blood, a fraction of peripheral blood comprising monocytes or a fraction of umbilical cord blood comprising monocytes. In one example a biological sample isolated form a subject may be enriched for monocytes. Alternatively the biological sample obtained from a subject may be depleted of monocytes.

In yet another example, a biological sample may comprise the substrate ECs from a subject. For example, the biological sample may be enriched for ECs.

### 1. Monocyte Depletion/Enrichment

Monocytes can be removed from a sample according to any method known in the art. In one example, the monocytes may be depleted from a sample (e.g., cells, tissue, surgical or biopsy specimens, plasma, serum, urine or other biological or non-biological sample as defined above) by cross-linkage of unwanted cells. For example, monocytes may be depleted from whole blood samples by RosetteSep® procedure (StemCell Technologies) according to manufacturer's protocol. Briefly, whole blood samples are diluted with an equal volume of PBS and 2% FBS and incubated with combination of mouse and rat monoclonal antibodies directed against cell surface antigens on human hematopoietic cells (e.g., CD36) and antigens on the red blood cells (RBCs) e.g., glycophorin A. The unwanted monocytes are cross-linked to RBCs forming immuno-rosettes with tetrameric antibody complexes. This increases the density of unwanted (rosetted) monocytes, such that they pellet along with free red blood cells (RBCs) when centrifuged over a buoyant density medium such as for example Ficoll-Paque® (StemCell Technologies). Desired components of the sample are generally not labelled with antibody (i.e., unrosetted) and are collected as highly enriched population at the interphase between the plasma and the buoyant density medium.

Alternatively, samples may be enriched for monocytes. According to this example, unwanted cells (other than monocytes) are cross-linked with antibodies monoclonal antibodies and the rosetted with RBCs, while the monocytes, which are not labelled and are collected as highly enriched population at the interphase between the plasma and the buoyant density medium.

In another example, monocytes are depleted from lymphocyte preparations, using magnetic beads, essentially as described by Siegel and Klohe et al., Scientific Communications. ASHIQuarterly. Fourth Quarter 2000, pp 87-89. Briefly, Dynabeads™ are magnetized polystyrene beads coated with mouse IgM monoclonalantibodies directed against selected CD membrane antigens on target cell populations. CD15 beads bind monocytes (to a variable degree). CD14 beads (selecting positive cells) bind monocytes that may not be completely removed by treatment with CD15 beads. Peripheral blood mononuclear cells (PBMCs) can be purified by negative selection of lymphocytes following magnet-assisted removal of the Dynabead™: monocyte complexes. PBMCs from whole blood are separated by Ficoll density gradient centrifugation. After removal of platelets and lysis of red cells, cell preparations are evaluated microscopically to determine the degree of monocyte contamination. If bead treatment is necessary, the PBMC preparation is pelleted by centrifugation, then resuspended e.g., using a RPMI comprising fetal calf serum and sodium citrate, CD14 and/or CD15 Dynabeads™ added for a time and under conditions sufficient for binding to occur, and a lymphocyte-enriched supernatant is collected. Dynabead™ monocyte complexes are discarded.

### Candidate Compositions or Compounds for Screening and Therapy

For the screening of compositions in the assay formats of the present invention, a peptidyl compound, antibody, small molecule or nucleic acid is screened to determine an ability thereof to modulate the production of non-haematopoietic cells from monocytes, or to modulate the ability of monocyte-derived non-haematopoietic cells to incorporate into an endothelial cell layer. The composition being assayed may modulate these effects by binding to a monocyte, serum factor(s), or to an endothelial cell or to endothelium. For example, compositions may interact with one or more monocyte receptors to thereby modulate processes that promote differentiation into non-haematopoietic cells. Alternatively or in addition, a composition may prevent interaction between a monocyte and a serum-based factor, or mimic a serum-based factor (e.g., an apolipoprotein) that normally interacts with one or more endothelial cell receptors to thereby modulate its incorporation into endothelium and/or modulate interaction between monocytes into non-haematopoietic cells. The present invention encompasses all such modes of action without limitation.

### Peptide Compounds

In one embodiment of the invention, a composition screened or assayed using the method of the invention comprises a peptidyl compound.

Such a peptidyl compound may be produced using any means known in the art. For example, a peptidyl compound is produced synthetically. Synthetic peptides are prepared using known techniques of solid phase, liquid phase, or peptide condensation, or any combination thereof, and can include natural and/or unnatural amino acids. Amino acids used for peptide synthesis may be standard Boc amino acid resin with the de-protecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino et al., Han, J. Org. Chem., 37:3403-3409, 1972. Both Fmoc- and Boc-protected amino acids can be obtained from various commercial sources, such as, for example, Fluka, Bachem, Advanced Chemtech, Sigma, Cambridge Research Biochemical, Bachem, or Peninsula Labs.

Alternatively, a synthetic peptide is produced using a technique known in the art and described, for example, in Stewart and Young (In: Solid Phase Synthesis, Second Edition, Pierce Chemical Co., Rockford, Ill. (1984) and/or Fields and Noble (Int. J. Pept. Protein Res., 35:161-214, 1990), or using an automated synthesizer. Accordingly, peptides of the invention may comprise D-amino acids, a combination of D- and L-amino acids, and various unnatural amino acids (e.g., methyl amino acids) to convey special properties. Synthetic amino acids include ornithine for lysine, fluorophenylalanine for phenylalanine, and norleucine for leucine or isoleucine.

In another example, a peptidyl compound is produced using recombinant means. For example, an oligonucleotide or other nucleic acid is placed in operable connection with a promoter. Methods for producing such expression constructs, introducing an expression construct into a cell and expressing and/or purifying the expressed peptide, polypeptide or protein are known in the art and described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

The term “promoter” is to be taken in its broadest context and includes the transcriptional regulatory sequences of a genomic gene, including the TATA box or initiator element, which is required for accurate transcription initiation, with or without additional regulatory elements (i.e., upstream activating sequences, transcription factor binding sites, enhancers and silencers) which alter gene expression in response to developmental and/or external stimuli, or in a tissue specific manner. In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters can contain additional copies of one or more specific regulatory elements to further enhance expression and/or alter the spatial expression and/or temporal expression of said nucleic acid molecule.

Placing a nucleic acid molecule under the regulatory control of, i.e., “in operable connection with”, a promoter sequence means positioning said molecule such that expression is controlled by the promoter sequence, generally by positioning the promoter 5′ (upstream) of the peptide-encoding sequence.

Suitable promoters and/or vectors for expressing a peptide will be apparent to the skilled artisan. For example, a typical promoters suitable for expression in a mammalian cell, mammalian tissue or intact mammal include, for example a promoter selected from the group consisting of, a retroviral LTR element, a SV40 early promoter, a SV40 late promoter, a cytomegalovirus (CMV) promoter, a CMV IE (cytomegalovirus immediate early) promoter, an EF1 promoter (from human elongation factor 1), an EM7 promoter or an UbC promoter (from human ubiquitin C).

A vector comprising such a suitable promoter is often used in an expression vector. A suitable expression vector for expression in a mammalian cell will be apparent to the skilled person and includes, for example, the pcDNA vector suite supplied by Invitrogen, the pCI vector suite (Promega), the pCMV vector suite (Clontech), the pM vector (Clontech), the pSI vector (Promega) or the VP16 vector (Clontech).

Typical promoters suitable for expression in viruses of bacterial cells and bacterial cells include, but are not limited to, the lacz promoter, the Ipp promoter, temperature-sensitive λL or λR promoters, T7 promoter, T3 promoter, SP6 promoter or semi-artificial promoters such as the IPTG-inducible tac promoter or lacUV5 promoter.

A number of other gene construct systems for expressing the nucleic acid fragment of the invention in bacterial cells are well-known in the art and are described for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have been described, such as for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO (Invitrogen, Carlsbad, Calif.); the pFLEX series of expression vectors (Pfizer Inc., CT, USA); the pQE series of expression vectors (QIAGEN, CA, USA), or the pL series of expression vectors (Invitrogen), amongst others.

Methods for producing an expression construct will be apparent to the skilled artisan and/or described, for example, in Sambrook et al (In: Molecular cloning, A laboratory manual, second edition, Cold Spring Harbor Laboratory, Cold Spring Harbor, N.Y., 1989).

Following construction of a suitable expression construct, said construct is introduced into a cell and the cell maintained for a time and under conditions sufficient for the expression of a peptide encoded by the nucleic acid. Optionally, the peptide is expressed as a fusion protein with a tag, e.g., a hexa-his tag or a myc tag to facilitate purification of the peptide, e.g., by affinity purification.

Alternatively, the peptide, polypeptide or protein is expressed using a cell free system, such as, for example, the TNT system available from Promega. Such an in vitro translation system is useful for screening a peptide library by, for example, ribosome display, covalent display or mRNA display.

In another example, a peptide library is screened to identify or isolate a compound capable of modulating production of non-haematopoietic cells such as for example, ECs or EC-like cells from monocytes in a cell culture and/or in an appropriate animal model. Alternatively or in addition, such screens are performed to identify or isolate a compound or a fragment thereof capable of promoting and/or enhancing vascular maintenance and/or repair and/or vascular network formation and/or development. By “peptide library” is meant a plurality of peptides that may be related in sequence and/or structure or unrelated (e.g., random) in their structure and/or sequence. Suitable methods for production of such a library will be apparent to the skilled artisan and/or described herein.

For example, a random peptide library is produced by synthesizing random oligonucleotides of sufficient length to encode a peptide of desired length, e.g., 7 or 9 or 15 amino acids. Methods for the production of an oligonucleotide are known in the art. For example, an oligonucleotide is produced using standard solid-phase phosphoramidite chemistry. Essentially, this method uses protected nucleoside phosphoramidites to produce a short oligonucleotide (i.e., up to about 80 nucleotides). Typically, an initial 5′-protected nucleoside is attached to a polymer resin by its 3′-hydroxy group. The 5′ hydroxyl group is then de-protected and the subsequent nucleoside-3′-phosphoramidite in the sequence is then coupled to the de-protected group. The internucleotide bond is then formed by oxidising the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988). Each of the oligonucleotides is then inserted into an expression construct (in operable connection with a promoter) and introduced into a cell or animal. Suitable methods for producing a random peptide library are described, for example, in Oldenburg et al., Proc. Natl. Acad. Sci. USA 89:5393-5397, 1992; Valadon et al., J. Mol. Biol., 261:11-22, 1996; Westerink Proc. Natl. Acad. Sci. USA., 92:4021-4025, 1995; or Felici, J. Mol. Biol., 222:301-310, 1991.

Optionally, the nucleic acid is positioned so as to produce a fusion protein, wherein the random peptide is conformationally constrained within a scaffold structure, eg., a thioredoxin (Trx) loop (Blum et al. Proc. Natl. Acad. Sci. USA., 97, 2241-2246, 2000) or a catalytically inactive staphylococcal nuclease (Norman et al, Science, 285, 591-595, 1999), to enhance their stability. Such conformational constraint within a structure has been shown, in some cases, to enhance the affinity of an interaction between a random peptides and its target.

To facilitate screening of a large number of peptides (i.e., to avoid producing a number of animals capable of expressing a peptide to be screened) a peptide may optionally be fused or conjugated to a protein transduction domain to facilitate its uptake into a cell in vitro or in vivo as the context requires. A suitable protein transduction domain will be apparent to the skilled person and includes, for example, HIV-1 TAT or polyarginine. Protein transduction domains are reviewed, for example, in Deitz and Bahr, Mol. Cell Neurosci. October; 27: 85-131, 2004.

### Antibodies

The present invention additionally provides for screening of antibodies and antibody libraries to thereby identify or isolate antibody-based compounds or fragments thereof capable of modulating production of non-haematopoietic cells such as for example, ECs or EC-like cells from monocytes in a cell culture and/or in an appropriate animal model. Alternatively or in addition, antibodies and antibody fragments are screened to identify or isolate an antibody or a fragment thereof capable of promoting and/or enhancing vascular maintenance and/or repair and/or vascular network formation and/or development.

By “antibody-based compound” is meant an antibody or a fragment or a derivative thereof, whether produced using standard or recombinant techniques. Accordingly, an antibody-based compound includes, for example, an intact monoclonal or polyclonal antibody, an immunoglobulin (IgA, IgD, IgG, IgM, IgE) fraction, a humanized antibody, or a recombinant single chain antibody (SCAb), as well as a fragment of an antibody, such as, for example Fab, F(ab)2, and Fv fragments, or a derivative of an antibody such as a humanized antibody or chimeric antibody or a fragment thereof produced by affinity maturation and/or recombination of or between heavy and/or light chains.

In one example, an antibody-based compound will be directed against a monocyte antigen, fragment thereof or a B cell epitope thereof.

In another example, an antibody-based compound will be directed against a serum factor e.g., an apolipoprotein, or mimic the effect of a serum factor on the differentiation process and/or incorporation of ECs into endothelium.

In yet another example, an antibody-based compound will be directed against an endothelial cell antigen, fragment thereof or a B cell epitope thereof.

Such an antibody, preferably a monoclonal antibody, is produced by immunizing an animal (e.g., a mouse) with the relevant immunogen. Optionally, the immunogen is injected in the presence of an adjuvant, such as, for example Freund's complete or incomplete adjuvant, lysolecithin and/or dinitrophenol to enhance the immune response to the immunogen. The immunogen may also be linked to a carrier protein, such as, for example, BSA. Spleen cells are then obtained from the immunized animal. The spleen cells are then immortalized by, for example, fusion with a myeloma cell fusion partner, preferably one that is syngeneic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification using the immunogen used to immunize the animal to isolate an antibody capable of binding thereto. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

Alternatively, an antibody library and preferably a library of antibodies that are expressed within a cell is used for the screening method of the invention. Such an antibody, also known as an intrabody, is essentially as recombinant ScFv antibody fragment. An ScFv antibody fragment is a recombinant single chain molecule containing the variable region of a light chain of an antibody and the variable region of a heavy chain of an antibody, linked by a suitable, flexible polypeptide linker. A library of ScFv fragments is produced, for example, by amplifying a variable region of a large and/or small chain from nucleic acid amplified from spleen cells that are derived from a subject (e.g., a mouse) that optionally, has been previously immunized with an immunogen of interest. These regions are cloned into a vector encoding a suitable framework including a linker region to facilitate expression of an intrabody that is stable when expressed in a cell (for example, see Worn et al., J. Biol. Chem., 275: 2795-803, 2003). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell. Biol. 15:1182-91, 1995; Lener et al., Eur. J. Biochem. 267:1196-205 2000).

### Nucleic Acids

The present invention additionally provides for screening of nucleic acids to identify or isolate a nucleic acid capable of modulating production of non-haematopoietic cells such as for example, ECs or EC-like cells from monocytes in cell culture and/or animal models. Alternatively or in addition, such screens are performed to identify or isolate a nucleic acid capable of enhancing vascular maintenance and/or repair and/or vascular network formation and/or development.

Preferably, a library of nucleic acids that are capable of being expressed in or entering a cell is screened.

Preferred nucleic acid-based compounds are antisense, ribozyme, RNAc, siRNA or aptamer compounds.

Anti-sense compounds are oligonucleotides comprising DNA or RNA or a derivative thereof (e.g., PNA or LNA) that is complementary to at least a portion of a specific nucleic acid target. Preferably, an antisense molecule comprises at least about 15 or 20 or 30 or 40 nucleotides complementary to the nucleotide sequence of a target nucleic acid. The use of antisense methods is known in the art (Marcus-Sakura, Anal. Biochem. 172: 289, 1988).

A ribozyme is an antisense nucleic acid molecule that is capable of specifically binding to and cleaving a target nucleic acid e.g., mRNA of a gene encoding a protein involved in monocytes differentiation into non-haematopoietic cells. A ribozyme that binds to a target nucleic acid and cleaves this sequence reduces or inhibits the translation of said nucleic acid. Five different classes of ribozymes have been described based on their nucleotide sequence and/or three dimensional structure, namely, Tetrahymena group I intron, Rnase P, hammerhead ribozymes, hairpin ribozymes and hepatitis delta virus ribozymes. Generally, a ribozyme comprises a region of nucleotides (e.g., about 12 to 15 nucleotides) that are complementary to a target sequence.

An RNAi (or siRNA or small interfering RNA) is a double stranded RNA molecule that is identical to a specific gene product e.g., mRNA of a gene encoding a protein involved in monocytes differentiation into non-haematopoietic cells. The dsRNA when expressed or introduced into a cell induces expression of a pathway that results in specific cleavage of a nucleic acid highly homologous to the dsRNA.

RNAi molecules are described, for example, by Fire et al., Nature 391: 806-811, 1998, and reviewed by Sharp, Genes and Development, 13: 139-141, 1999). As will be known to those skilled in the art, short hairpin RNA (“shRNA”) is similar to siRNA. However, the shRNA molecule comprises a single strand of nucleic acid with two complementary regions separated by an intervening hairpin loop such that, following introduction to a cell, it is processed by cleavage of the hairpin loop into siRNA.

A preferred siRNA or shRNA molecule comprises a nucleotide sequence that is identical to about 19-21 contiguous nucleotides of the target mRNA. Preferably, the target sequence commences with the dinucleotide AA, comprises a GC-content of about 30-70% (preferably, 30-60%, more preferably 40-60% and more preferably about 45%-55%), and is specific to the nucleic acid of interest.

A nucleic acid aptamer (adaptable oligomer) is a nucleic acid molecule that is capable of forming a secondary and/or tertiary structure that provides the ability to bind to a molecular target e.g., mRNA of a gene encoding a protein involved in monocytes differentiation into non-haematopoietic cells. Suitable methods for producing and/or screening an aptamer library is described, for example, in Ellington and Szostak, Nature 346:818-22, 1990.

Generally, an aptamer is identified from a library of nucleic acids that comprise at least a random region are screened. For example, the library is screened to identify an aptamer that is capable of modulating angiogenic potential and/or activity and/or capable of modulating neovascularization potential and/or activity, or alternatively or in addition, capable of enhancing vascular network formation and/or development.

An aptamer usually comprises at least one random region of nucleic acid, flanked by a known sequence that may provide the annealing site for hybridization of a PCR primer for later amplification of the aptamer and/or may provide for conformational constraint, e.g., by forming a hairpin. The random sequence portion of the oligonucleotide can be of any length (however, is generally 30 to 50 nucleotides in length) and can comprise ribonucleotides and/or deoxyribonucleotides and can include modified or non-natural nucleotides or nucleotide analogs. Random oligonucleotides can be synthesized from phosphodiester-linked nucleotides using solid phase oligonucleotide synthesis techniques well known in the art and/or described herein. Typical syntheses carried out on automated DNA synthesis equipment yield 1015-1017 molecules.

Due to the large number of aptamers that may be screened, a screening assay described herein may be performed using arrays of aptamers, and those arrays capable of modifying phenotype rescreened to determine the specific aptamer of interest.

### Small Molecules

The present invention additionally provides for the screening of small molecules and small molecule libraries to thereby identify or isolate a small molecule capable of modulating production of non-haematopoietic cells such as for example, ECs or EC-like cells from monocytes in a cell culture and/or in an appropriate animal model. Alternatively or in addition, such screens are performed to thereby identify or isolate a small molecule capable of promoting and/or enhancing vascular maintenance and/or repair and/or vascular network formation and/or development.

The structures of small molecules vary as do methods for their syntheses. The present invention contemplates the screening without limitation of any small molecule or small molecule library.

For example, U.S. Pat. No. 6,168,192 describes a multidimensional chemical library that comprises converting a set of at least two different allyl carbonyl monomers to form monomer derivatives by converting the allyl group to another group and covalently linking at least two of the produced monomers to form oligomers. McMillan et al., (Proc Natl Acad Sci USA. 97: 1506-1511, 2000) describes an encoded chemical library produced by the ECLiPS method, wherein the compounds of the library are based on a pyrimidineimidazole core prepared on polyethylene glycol-grafted polystyrene support. To produce the library, compounds are attached to resin by a photolabile o-nitrobenzyl amide linker, primary amines are introduced and acylated using fluorenylmethoxycarbonyl (Fmoc)-protected amino acids and deprotected according to an fmoc synthesis protocol and the resulting free amines heteroarylated by electrophilic substitution with a set of nine substituted pyrimidines. The library produced comprises 8,649 compounds Alternatively, or in addition, small molecule libraries may comprise statin esters (e.g., U.S. Pat. No. 6,255,120), monomycin analogs (e.g., U.S. Pat. No. 6,207,820), fused 2,4-pyrimidinediones (e.g., U.S. Pat. No. 6,025,371), dihydrobenzopyran based molecules (e.g., U.S. Pat. No. 6,017,768), 1,4-benzodiazepin-2,5-dione based compounds (e.g., U.S. Pat. No. 5,962,337), benzofuran derivatives (e.g., U.S. Pat. No. 5,919,955), indole derivatives (e.g., U.S. Pat. No. 5,856,496), polyketides products (e.g., U.S. Pat. No. 5,712,146), morpholino compounds (e.g., U.S. Pat. No. 5,698,685) or sulphonamide compounds (e.g., U.S. Pat. No. 5,618,825). Combinations of such libraries and other libraries known to the skilled artisan may also be employed.

High throughput analytical/predictive techniques, such as, for example, sequential high throughput screening (SHTS) may be employed to select classes of compounds having a particular activity, based on the activity of a known agonist or antagonist and the structure-activity relationship (SAR) exhibited by the known agonist or antagonist. For example, SHTS is an iterative process comprising screening a library of compounds for activity and selecting a sub-set of compounds for subsequent screening wherein selection is driven by structure-activity relationships (SARs) within the screened compounds and using those relationships to drive further selection.

Recursive partitioning (RP) can be used in conjunction with high-throughput screening techniques, such as, SHTS, by identifying relationships between specific chemical structural features of the molecules and biological activity. The premise of RP is that the biological activity of a compound is a consequence of its molecular structure. Accordingly, it is useful to identify those aspects of molecular structure that are relevant to a particular biological activity. By gaining a better understanding of the mechanism by which the compound acts, additional compounds for screening can more accurately be selected. Suitable RP methods are described, for example in Hawkins, D. M. and Kass, G. V., (In: Automatic Interaction Detection. In Topics in Applied Multivariate Analysis; Hawkins, D. H., Ed.; 1982, Cambridge University Press, pp. 269-302).

Quantitative structure activity relationship (QSAR) is also useful for determining a feature or features of a compound required for or useful for a desired biological activity. QSAR models are determined using sets of compounds whose molecular structure and biological activity are known, a training set. QSAR approaches are either linear or nonlinear. The linear approach assumes that the activity varies linearly with the level of whatever features affect it, and that there are no interactions among the different features.

Nonlinear QSAR approaches account for the fact that activity can result from threshold effects; a feature must be present for at least some threshold level for activity to occur. Furthermore, as interactions between features are observed in many QSAR settings, the utility of one feature depends upon the presence of another. For example, activity may require the simultaneous presence of two features.

### Production of Compounds

In one example, the present invention additionally provides a process of producing a composition capable of modulating production of non-haematopoietic cells such as for example, ECs or EC-like cells from monocytes, alternatively or in addition providing a composition capable of enhancing vascular maintenance and/or repair and/or vascular network formation and/or development, said process comprising performing one of more of the following:
i) determining the structure of an isolated or identified composition;
ii) providing the isolated or identified composition or the structure of the isolated or identified composition;
iii) synthesizing the isolated or identified composition; or
iv) obtaining the isolated or identified composition.

As used herein, the term “providing the composition” shall be taken to include any chemical or recombinant synthetic means for producing said composition (with or without derivitization) or alternatively, the provision of a composition that has been previously synthesized by any person or means. Suitable methods for producing a composition are described herein or known in the art.

As used herein, the term “synthesizing the composition” shall be taken to include any chemical or recombinant synthetic means for producing said composition.

### Formulations

The present invention clearly contemplates the use of modulators identified or isolated in any screen performed as described according to any example hereof in the preparation of a composition for use in therapy e.g., in the preparation of a composition for the treatment or prophylaxis of atherosclerosis and/or one or more vascular complications in a human or other animal subject.

Accordingly, the invention provides a pharmaceutical composition or vaccine comprising a modulator of non-haematopoietic cell production from monocytes and/or incorporation of such non-haematopoietic cells e.g., ECs and/or EC-like cells into endothelium. Preferred pharmaceutical formulations comprise the modulator in combination with a pharmaceutically acceptable diluent.

The modulator is conveniently formulated in a pharmaceutically acceptable carrier, excipient or diluent.

Suitable diluents or excipients include, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the pharmaceutical composition are adjusted according to routine skills in the art.

A pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material. In powders, the carrier is a finely divided solid which is in a mixture with the finely divided active component. In tablets, the active component is mixed with the carrier having the necessary binding capacity in suitable proportions and compacted to the desire shape and size. Powders and tablets may contain varying percentage amounts of an active compound. A representative amount in a powder or tablet may contain from 0.5 to about 90 percent of the active compound; however, a skilled pharmacist will know when other amounts are required or advisable. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Liquid form preparations include solutions, suspensions, slurries and emulsions, for example, water or water-propylene glycol solutions. For example, liquid preparations for parenteral injection can be formulated as solutions in aqueous polyethylene glycol solution. Injectable preparations, for example, sterile injectable aqueous or oleaginous suspensions may be formulated according to the known art using suitable dispersing or wetting agents and suspending agents. An injectable preparation may also be a sterile injectable solution or suspension in a nontoxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The modulator may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the modulators are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration as an aerosol can be prepared by processes well-known to the person skilled in the art. For their preparation, for example, solutions or dispersions of the modulator in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

Tablets or capsules for oral administration and liquids for intravenous administration are preferred compositions.

The modulator may also be administered in the form of a liposome. Liposomes are microscopic vesicles that consist of one or more lipid bilayers surrounding aqueous compartments. (Bakker-Woudenberg et al., Eur. J. Clin. Microbiol. Infect. Dis. 12(Suppl. 1), S61 (1993); and Kim, Drugs 46, 618 (1993)). Liposomes are similar in composition to cellular membranes and as a result, liposomes generally are administered safely and are biodegradable.

Techniques for preparation of liposomes and the formulation (e.g., encapsulation) of various molecules, including peptides and oligonucleotides, with liposomes are well known to the skilled artisan.

Depending on the method of preparation, liposomes may be unilamellar or multilamellar, and can vary in size with diameters ranging from 0.02 μm to greater than 10 μm. A variety of agents are encapsulated in liposomes. Hydrophobic agents partition in the bilayers and hydrophilic agents partition within the inner aqueous space(s) (Machy et al., LIPOSOMES IN CELL BIOLOGY AND PHARMACOLOGY (John Libbey 1987), and Ostro et al., American J. Hosp. Pharm. 46, 1576 (1989)).

Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446, 368 (1985)). In the present context, the modulator may be localized on the surface of the liposome. Irrespective of the mechanism or delivery, however, the result is the intracellular disposition of the modulator.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides or lipopeptides.

Cationic liposome preparations are made by conventional methodologies (Feigner et al, Proc. Nat'l Acad. Sci USA 84, 7413 (1987); Schreier, Liposome Res. 2, 145 (1992)). Commercial preparations, such as Lipofectin (Life Technologies, Inc., Gaithersburg, Md. USA), are readily available. The amount of liposomes to be administered are optimized based on a dose response curve Feigner et al., supra.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. (See COLLOIDAL DRUG DELIVERY SYSTEMS, vol. 66, J. Kreuter, ed., Marcel Dekker, Inc. 1994).

Other forms of delivery particle, for example, microspheres and the like, also are contemplated for delivery of the modulator.

Nucleic acid-based modulators may be cloned into a suitable vector (eg. vaccinia, canary pox, adenovirus, or other eukaryotic virus vector). Preferably, nucleic acid modulators are formulated into a suitable vector for delivery.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Guidance in preparing suitable formulations and pharmaceutically effective vehicles, are found, for example, in REMINGTON'S PHARMACEUTICAL SCIENCES, chapters 83-92, pages 1519-1714 (Mack Publishing Company 1990) (Remington's), which are hereby incorporated by reference.

The present invention also contemplates any mode of administration of a medicament or formulation as described herein e.g., parenteral injection by the sub-cutaneous, intravenous, intra-arterial, intraocular route, or by inhalation, or by ingestion including a sub-lingual route, or by topical administration. Combinations of different administration routes are also encompassed.

For example, inhalable compositions are generally administered in an aqueous solution e.g., as a nasal or pulmonary spray. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering the solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; and in U.S. Pat. No. 4,778,810 (each incorporated herein by reference). Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Mucosal formulations are administered as dry powder formulations e.g., comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5 micron mass median equivalent aerodynamic diameter (MMEAD), commonly about 1 micron MMEAD, and more typically about 2 micron MMEAD. Maximum particle size appropriate for deposition within the nasal passages is often about 10 micron MMEAD, commonly about 8 micron MMEAD, and more typically about 4 micron MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

Standard methods are used to administer injectable formulations of the present invention.

The present invention will now be described by way of the following non-limiting examples and the accompanying drawings.

### Example 1

### Materials and Methods

### 1.1 Co-Culture of ECs and Peripheral Blood Mononuclear Cells

Human umbilical vein ECs (HUVEC) were isolated from human umbilical veins using collagenase digestion. Briefly, umbilical cords from normal or caesarean deliveries are collected and perfused with Ca²⁺ and Mg²⁺-free phosphate buffer saline (PBS). HUVECs were removed by short treatment at 37° C. with collagenase solution (0.1% in PBS with Ca²⁺ and Mg²⁺). HUVECS were cultured and expanded for use in investigation at no later than passage 4. Cells were grown under standard culture conditions of 37° C. in a 95% air/5% CO₂ atmosphere in M199 media (Cell Applications or Invitrogen Australia) supplemented with 10% foetal bovine serum (FBS; Trace Bioscience, or Invitrogen Australia) or with 10% heat-inactivated human serum and with a D-glucose concentration of 5.0 mM. The human serum was obtained from healthy donors within 7 days of the experiment. The human serum was filtered with 0.22 μm PES membrane (Millipore) and heat inactivated at 56° C. for 30 minutes.

Alternatively, human coronary artery ECs (HACECs) are cultured and expanded for use in the method of the invention. HACECs are cultured and expanded for use in investigation at no later than passage 4. Cells are grown under standard culture conditions at 37° C. in a 95% air/5% CO₂ atmosphere in HCAEC growth media (Cell Applications) supplemented with 10% foetal bovine serum (FBS; Trace Bioscience or Invitrogen Australia) or with 10% heat-inactivated human serum as above and with a D-glucose concentration of 5.0 mM.

The ECs were screened for human leucocyte antigen-A2 type (HLA-A2) expression with direct immunofluorescence and flow cytometry (Beckman Coulter). The batches of HUVEC cells that were HLA-A2 negative were used in these investigations. Alternatively, batches of HACECs that are HLA-A2 negative are used in these investigations. The batches of HLA-A2 negative HUVECs preferably at second passage were grown to approximately 75% sub-confluence on 6-wells cell culture plates (Becton Dickinson Labware, growth area 9.6 cms² per well) at a density varying from 10,000 to 500,000 cells per well 24 hours before the co-culture. Usually a density of 500,000 cells per well is needed to achieve a fully confluent layer of HUVECS. Alternatively, batches of HLA-A2 negative HCAECs preferably at second passage are grown to approximately 75% sub-confluence on 6-wells cell culture plates (Bectin Dickinson Labware).

Fresh peripheral blood mononuclear cells (PBMCs) were obtained from venous blood of healthy HLA-A2 positive donors (n=5) by gradient centrifugation on Lymphoprep in a hypertonic medium (Lymphoprep™) and immediately seeded onto HUVECs. Alternatively, the PBMCs are immediately seeded onto HCAECs layers at densities ranging from 0.25 to 2×10⁶ cells per well. The non-adherent cells were washed off after 2 to 3 hours of co-incubation by extensive washing with 4 ml of M199 per well and the cell cultures were maintained in M199 media supplemented with either 10% fetal bovine serum (Invitrogen Australia) or 10% heat-inactivated human serum obtained from healthy donors within 7 days of the experiment, which was prepared as described supra. The co-cultured cell layer containing HUVECs were detached at multiple time points between day 0 and day 6 by addition of 20 μl of detachment solution containing 0.12% trypsin and analyzed by direct immunofluoresence and flow cytometry inter alia for expression of cell surface markers. The PBMC derived cells were identified by HLA-A2 positive expression, thereby allowing to distinguish from the HLA-A2 negative ECs in the co-culture. To assess for VCAM-1 expression, the co-culture of PBMCs and HUVECs was stimulated with tumor necrosis factor-α (TNF-α) at a dose of 10 ng/ml for 24 hours. Alternatively, the co-culture of PBMCs and HCAECs is stimulated with TNF-α at a dose of 10 ng/ml for 24 hours.

### 1.2 Antibodies for Detecting Expression of Cell Surface Markers

Expression of cell surface markers was analyzed by two-colors direct immunofluoresence and flow cytometry (Cytomics FC500, Beckman Coulter). Alexa Fluor® and R-phycoerythrin (R-PE) conjugated primary antibodies used to identify cells in this study include mouse monoclonal AF488-conjugated anti-HLA-A2 (Serotec Inc. Raleigh, N.C.), mouse monoclonal RPE-conjugated anti-CD31 (BD Biosciences; Bedford, Mass.), anti-CD105 (Serotec Inc. Raleigh, N.C.), anti-CD144 (Santa Cruz Biotechnology), anti-CD14 (BD Biosciences; Bedford, Mass.), anti-CD11b (BD Biosciences), anti-CD36 (BD Biosciences), anti-CD115 (R&D Systems), anti-KDR (R&D System), anti-Tie-2 (BD Biosciences; Bedford, Mass.), anti-CD34 (BD Biosciences; Bedford, Mass.), anti-ICAM-1 (BD Biosciences; Bedford, Mass.), and anti-VCAM-1 (BD Biosciences; Bedford, Mass.). The isotype matched control antibodies recommended by the manufacturer were used for these investigations.

### 1.3 Immunofluoresence and Flow Cytometry Analysis

Detached cells were washed in PBS. The cells were counted manually using a haemocytometer. Aliquots containing 10⁵ cells in 500 μl of PBS were incubated at 4° C. for 45 minutes with 5-10 μl of saturating concentrations of monoclonal antibodies to HLA-A2, CD31, CD36, CD34, CD105, CD144, CD146, CD14, CD11b, CD115, KDR, Tie-2, ICAM-1, and VCAM-1 respectively, or with the isotype matched control antibodies. After three washes in PBS, quantitative fluorescence analysis was performed using a fluorescence activated cell analysis (FACS) Calibur Flow Fytometer and CellQuest software program (Becton Dickinson, USA). Histograms of cell number versus fluorescence intensity were recorded for at least 5000 cells per sample.

For the purpose of cell sorting, detached cells are treated and stained essentially as described above, except that cell sorting fluorescence analysis is performed using FACStar Plus Cell Sorter Flow Cytometer and CellQuest software program (Becton Dickinson, USA). Cells are sorted into in M199 media (Cell Applications) supplemented with 10% foetal bovine serum (FBS; Trace Bioscience) or with 10% heat-inactivated human serum and with a D-glucose concentration of 5.0 mM, either directly onto 96 well plates for further analysis or stored on ice until further use.

### 1.4 Preparation of Lipid Free Apolipoprotein A-I and Treatment of the Co-Culture

High-density lipoproteins (HDLs) were prepared from samples of expired human plasma (Gribbles Pathology, Adelaide, Australia) by sequential ultracentrifugation (1.07<density<1.21 g/ml). The HDLs were delipidated and apolipoprotein A-I (apoA-I) was isolated by anion exchange chromatography on a column of Q-Sepharose Fast Flow (Amersham Pharmacia Biotech) attached to a fast protein liquid System. The lipid-free apoA-I was dialyzed into endotoxin-free PBS and filtered with 0.22 μm PES membrane (Millipore) and added to the co-culture of PBMCs and ECs at concentrations of 0.25 mg/ml, 0.5 mg/ml, 1.0 mg/ml and 2.0 mg/ml. Co-culture controls without apoA-I received equal volumes of sterile PBS and the groups were compared using student t-test.

### 1.5 Preparation of Lipid-Free Apolipoprotein A-II and Treatment of the Co-Culture

Human high-density lipoproteins (HDLs) were isolated from samples of expired human plasma (Gribbles Pathology, Adelaide, Australia) by sequential ultracentrifugation (1.07<density<1.21 g/ml). The HDLs were delipidated and apolipoprotein A-II (apoA-II) was isolated by anion exchange chromatography on a column of Q-Sepharose Fast Flow (Amersham Pharmacia Biotech) attached to a fast protein liquid System. The lipid-free apoA-II was dialyzed into endotoxin-free PBS and filtered with 0.22 μm PES membrane (Millipore) and added to the co-culture of PBMCs and ECs at concentrations of 0.25 mg/ml and 0.4 mg/ml. Co-culture controls without apoA-II received equal volumes of sterile PBS and the groups were compared.

### 1.6 Preparation of LDL and Treatment of the Co-Culture

Human plasma low density lipoproteins (LDLs) were isolated from pooled samples of expired human plasma (Gribbles Pathology, Adelaide, Australia) by top fraction isolation obtained by sequential ultracentrifugation (density=1.063 g/ml) in the presence of 0.0570 EDTA according to the method of Gillies et al., J. Am. Chem. Soc. 78: 4103 (1956); and Gofman et al., Science. 111:166 (1950), as previously described in Janado et al., J. Lipid Res. 6: 331 (1965). A typical LDL preparation used in this study contains 80.6% lipid and 19.4% protein on a dry weight basis as determined by gravimetric methods Nishida, T., and H. Nishida. J. Biol. Chem. 240: 225 (1965).

### 1.7 Statistical Analysis

Immunofluoresence and the flow cytometry analysis results revealed a Gaussian distribution. Hence comparisons between any two groups were compared using two-tailed unpaired student t-test and results of P<0.05 were considered statistically significant.

### 1.8 In Vitro Angiogenic Assays

### 1.8.1 Cell Proliferation Assay

Cells are sorted onto 96 well plates at densities of 5000 cells per well in M199 media (Cell Applications) supplemented with 10% foetal bovine serum (FBS; Trace Bioscience) or with 10% heat-inactivated human serum and with a D-glucose concentration of 5.0 mM. Cell proliferation is determined by EdU incorporation using Click-iT™ EdU Cytometry Assay Kit according to manufacturer's protocol (Invitrogen).

### 1.8.2 Cell Migration Assay

Cell migration assays are performed in modified Boyden chambers in a 24 well plate format (Costar) with a polyvinyl 8 μm membrane. In brief, 1×10⁴ sorted cells are seeded in the upper well of the chamber in a 100 μL volume of serum free M199 media, 600 μL of M199 media supplemented with 10% foetal bovine serum (FBS; Trace Bioscience) or with 10% heat-inactivated human serum and with a D-glucose concentration of 5.0 mM is added to the lower chamber. After 18 hrs, 24 hrs and 48 hrs cells are removed from the upper chamber and the transwell is washed twice in PBS. Next the transwell is incubated for 30 mM in 600 μL of PBS containing 10 μg/mL Ulex-Lectin FITC conjugate (Sigma-Aldrich) at room temperature. The transwell is rinsed twice in PBS, the membrane removed and mounted in DAPI-containing aqueous VectaMount (Vector Laboratories). DAPI and FITC dual-positive cells are then visualized by fluorescent microscopy (Olympus), photomicrograhs are taken (DP Controller) and the number of HUVEC cells that migrate to the lower transwell side of the membrane per field is calculated.

### 1.8.3 Matrigel Tubulogenesis Assays

EC formation of vascular-like structures is assessed on growth factor-reduced Matrigel (Becton Dickinson) in a method described by Weis et al., Art. Thromb. Vasc. Biol. 22, 1817-1823 (2002). Approximately 30 μL of Growth Factor Reduced Matrigel (BD) is added to each well of a 96-well plate and allowed to coat for 1 hr at 37° C. under standard tissue culture incubator conditions. Next, 2×10³ of sorted cells are added to each well and tubule formation is observed over a period of 18 hrs, 24 hrs and 48 hrs. Photomicrographs are taken on the Olympus microscope and the tubule area and formation assessed using ImageJ Software (NIH).

### Example 2

### PBMCs Recruited by HUVEC were Circulating Monocytes

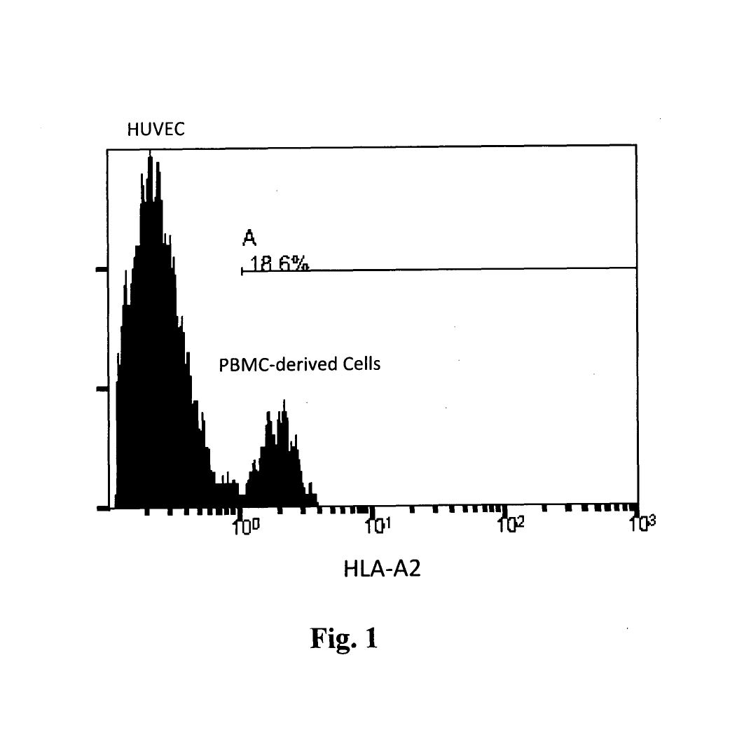

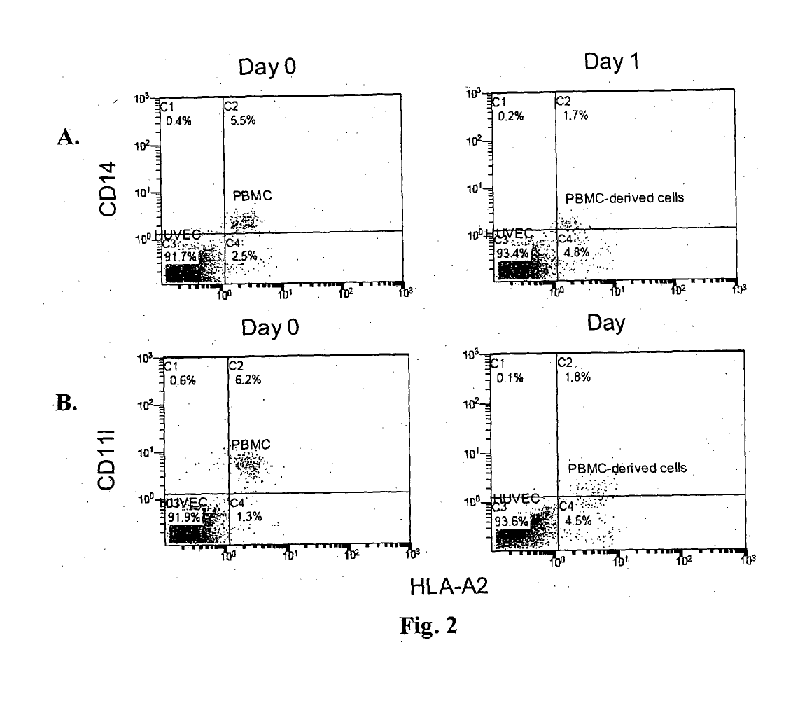

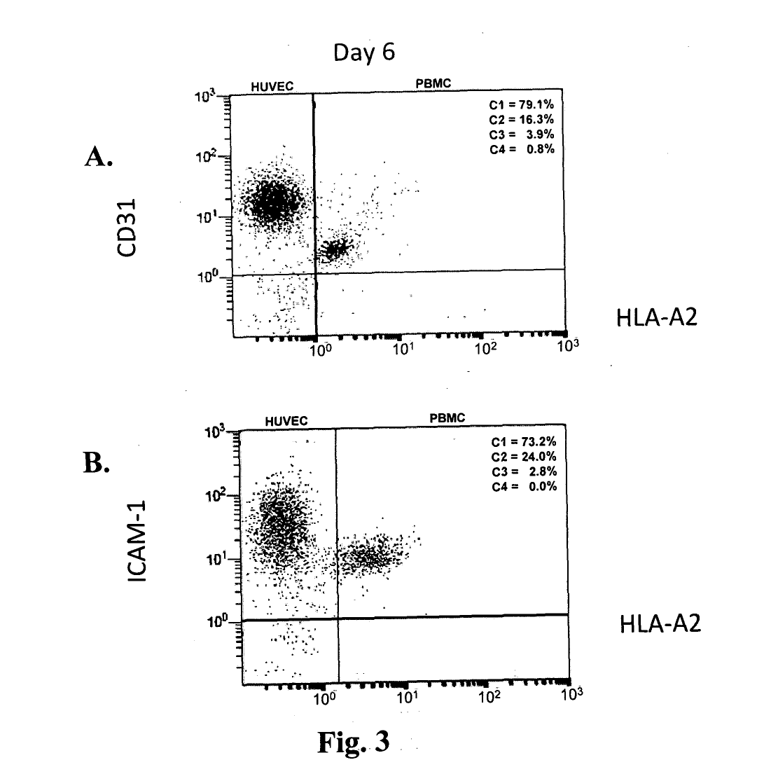

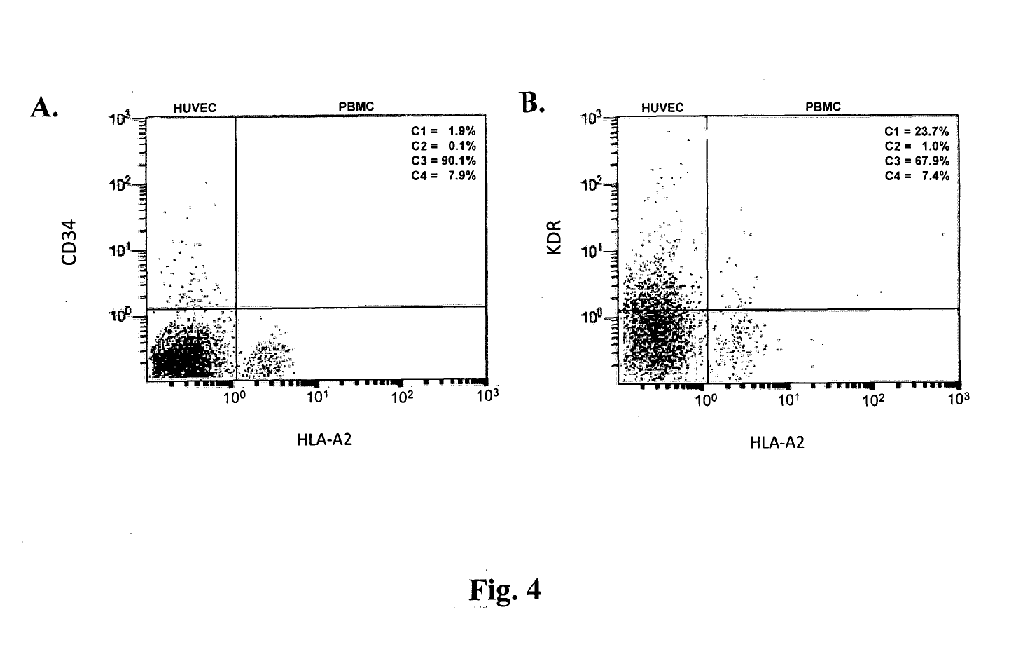

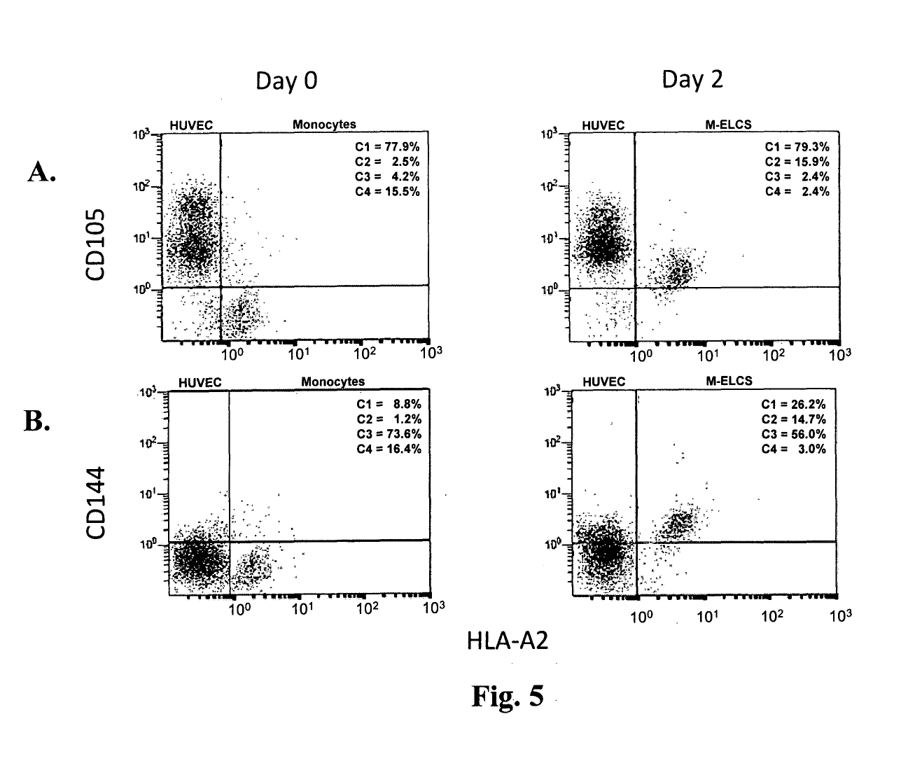

The inventor assessed the characteristics of the PBMCs that become recruited by the EC layer following contact and adherence of the PBMCs to the ECs, as shown in FIG. 1. The results illustrated that after contacting cells by co-culturing of HUVEC with PBMCs for 2-3 hours, and washing the non-adherent cells off, as described supra, two distinct populations of cells, HLA-A2 positive (PBMC) and HLA-A2 negative (HUVEC) cells were clearly distinguished using direct immunofluoresence and flow cytometry analysis (FIG. 1). Cells recruited by the HUVECs in the cell culture were the HLA-A2 positive adherent PBMCs. All the PBMCs recruited by the HUVECs initially expressed CD14, CD11b, CD31 and ICAM-1 (day 0) (FIG. 2 and FIG. 3). The endothelial progenitor markers CD34, KDR (FIG. 4) or Tie-2 (data not shown) were not expressed by the recruited PBMCs immediately after 2 hours of co-culture. Furthermore, the endothelial cell markers CD144 and CD105 were also not expressed by the PBMCs recruited by the HUVEC layer at this stage i.e., immediately after 2 to 3 hours of co-culture (day 0) (FIG. 5). Accordingly the phenotype of the adherent HLA-A2 positive PBMCs recruited by the HUVEC was consistent with that of circulating CD14 positive monocytes. Therefore the inventors concluded that monocytes are recruited by ECs following contact with the ECs.

### Example 3

### Monocytes Recruited into HUVEC Layer Rapidly Trans-Differentiated into EC-Like Cells

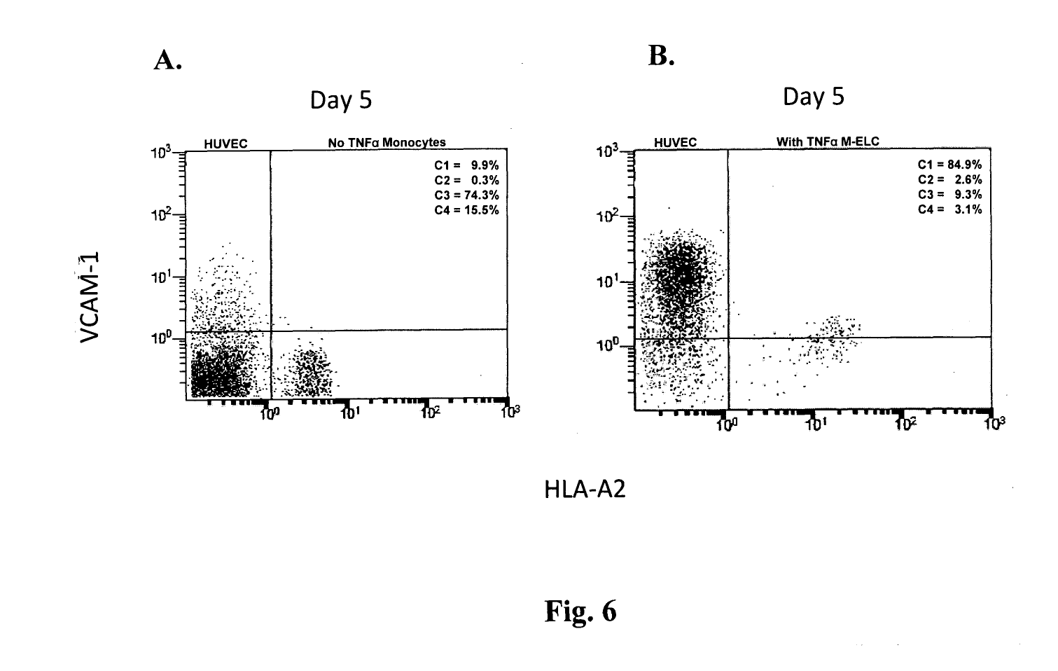

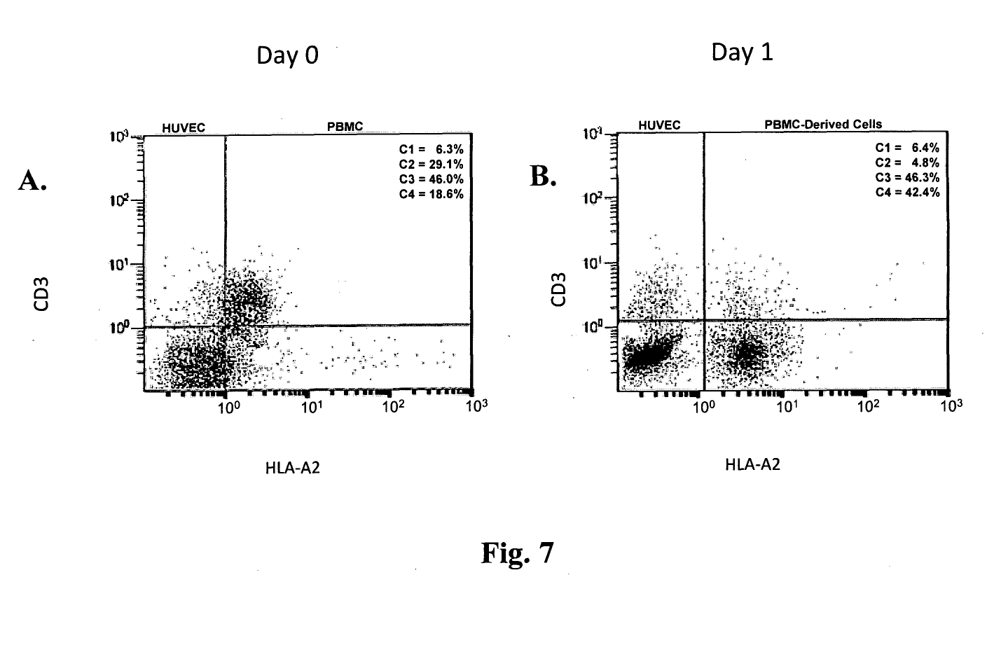

The inventor assessed the effects of co-culturing of HUVECs with PBMCs on the trans-differentiation of the recruited monocytes, as shown in FIG. 2, FIG. 3, FIG. 5, FIG. 6 and FIG. 7. The inventor co-cultured HUVEC with PBMCs for 2 to 3 hours followed by washing the non-adherent cells off and maintaining the co-culture up to 6 days, as described supra. The results illustrate that 3 day after cell contact and commencement of the co-culture of HUVECs with PBMCs there was down regulation of expression of CD14 in the HLA-A2 positive (monocyte-derived) cells that had been recruited and adhered to the endothelial layer (FIG. 2) and after two days in the co-culture following contact and adherence to the EC cells, these adherent monocyte-derived cells were expressing the EC surface markers CD105 and CD144 (FIG. 5). CD144 cell surface antigen is an essential component of endothelial tight junction and is a reflection of mature endothelial functionality. Specifically, at day 2, the proportion of HLA-A2 positive and CD105 positive monocyte-derived cells had risen to 15.9% of total cells in the co-culture, which was similar to the 15.5% HLA-A2 positive monocytes recruited by the HUVECs after 2 hours of co-culture, following washing of non-adherent PBMCs (i.e., at day 0). Similarly, at day 2, the proportion of HLA-A2 positive and CD144 positive monocyte-derived cells had risen to 14.7% of total cells in the co-culture, which was similar to the 16.4% HLA-A2 positive monocytes recruited by the HUVECs after contact of monocytes with ECs i.e., 2 hours of co-culture (day 0) (FIG. 5). Furthermore, the results also demonstrated that by day 3, the monocyte markers CD14 and CD11b and CD36 expression were down-regulated in the HLA-A2 positive monocyte-derived cells recruited by HUVEC at the commencement of the co-culture, after contact of monocytes with ECs (FIG. 2). Specifically, CD36 was initially expressed by the majority of the endothelial adherent PBMCs at 2 hours of co-culture (FIG. 7A). However the expression of CD36 was rapidly down-regulated after 24 hours of co-culture and CD36 was only expressed by 10% of the PBMC-derived cells (FIG. 7B). CD115 was not expressed by the HLA-A2 positive cells at any time up to 5 days of co-culture of PBMCs with ECs (data not shown). CD31 and ICAM-1 persisted to be expressed on the HLA-A2 positive (monocyte-derived cells) throughout the 6 days co-culture period (FIG. 3). When cells that had been in co-culture for 5 days were activated by TNFα (10 ng/ml for 24 hours) it was apparent that VCAM-1 was expressed by about half of the HLA-A2 positive cells (FIG. 6). Taken together, these phenotypic findings provide compelling evidence that the recruited HLA-A2 positive monocytes had rapidly trans-differentiated into functional endothelial-like cells, with detectable changes observed within 24 hours following contact with ECs.

Without being bound by mode of action or theory, the inventor reasoned that CD144 was a highly specific component of the endothelial tight junction that is essential for the endothelium to function as a barrier, the lack of which leads to endothelial leakage. Accordingly, the presence of CD144 provided a critical indicator to the inventor of endothelial functionality. Also without being bound by any mode of action or theory, the inventor reasoned that CD105 was an antigen expressed by endothelium in angiogenic mode. Accordingly, CD105 expression in combination with down-regulation of CD14 and/or CD11b and/or CD36 expression and a lack of CD115 expression provided compelling evidence to the inventor of an endothelial phenotype. Another observation in support of endothelial differentiation of the recruited substrate monocytes was the appearance of VCAM-1 by the monocyte-derived cells upon TNFα stimulation. Although VCAM-1 was not specific to endothelium, monocyte or macrophages were not known to express this adhesion molecule. The inventor also reasoned that CD31 and ICAM-1 were not useful in assessing monocyte differentiation in the method of the invention as both of these antigens may be expressed by endothelium, blood monocytes and macrophages.

### Example 4

### Quantification of Endothelial Incorporation of Monocyte-Derived EC-Like Cells

### 1. Quantification of HLA-A2 Positive Monocyte-Derived Endothelial-Like Cells

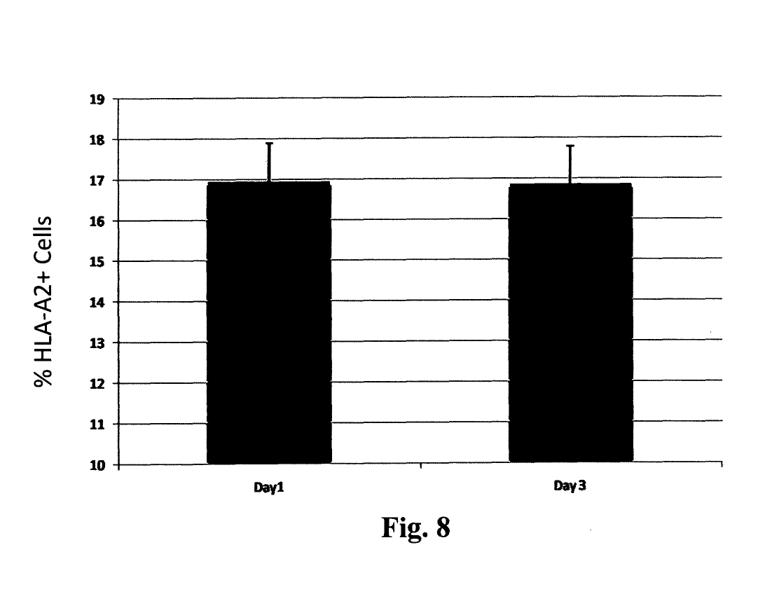

The inventor has quantified the proportion of the HLA-A2 positive monocyte-derived endothelial-like cells of the total cell population in the co-culture system, as illustrated in FIG. 8. Culturing PBMCs with HUVECs was repeated at least three (3) times and cells were characterized at day 1 and day 3 following washing of non-adherent PBMCs i.e., after contact of monocytes with ECs. The results illustrate that the proportion of these HLA-A2 positive cells remained relatively constant up to at least 3 days in co-culture (FIG. 8) (Student t test P value, not significant). Although, in one set of experiments the percentage of adherent HLA-A2 positive monocyte-derived EC-like cells recruited by the HUVEC and incorporated into the EC layer varied between 12 to 19% of the total cell population in the co-culture, with different batched of HUVEC. In another set of experiments, the percentage of HLA-A2 positive monocyte-derived EC-like cells in the co-culture varied between 8 to 25% with different batched of HUVEC. Accordingly a single batch of HUVECS was used for each experiment.

### 2. Effects of Varying the Seeding Density of PBMCs

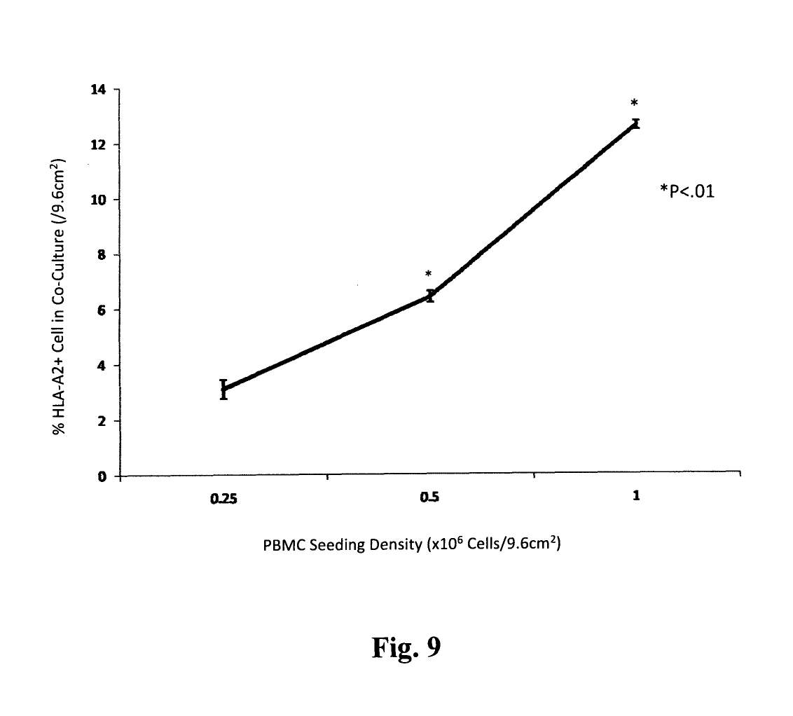

In one set of experiments the inventor assessed the effects of varying the seeding density of PBMCs prior to contact of the PBMCs with the ECs on the proportion of recruited monocytes that can trans-differentiated into EC-like cells, as observed by expression of cell surface markers during PBMC/HUVEC co-culture. PBMCs seeding densities of 0.25×10⁶ cells per well (low density), 0.5×10⁶ cells per well (intermediate density) and 1×10⁶ cells per well (high density) conditions were investigated. Six-wells cell culture plates (Bectin Dickinson Labware) were used with each well having 9.6 cm² plating surface area. For any given experiment, the EC layers were prepared from a single batch of HUVECs that were plated 24 hours before the experiment at a seeding density of 0.5×10⁵ cells per well to ensure confluent layers. The percentages of the endothelial-like cells after 24 hours of co-culture were 3.1±0.35% in the low density group, 6.3±0.2% in the intermediate density group and 12.6±0.15% in the high density group (FIG. 9). The monocyte-derived EC-like cells numbers estimated using these figures from the total cell counts were 20,000 cells per well in the low density group, 50,000 cells per well in the intermediate density group and 150,000 cells per well in the high density group. These represented 7.6%, 10.9% and 15% of the starting PBMC population respectively. Thus, the monocyte-derived EC-like cells generated by the recruited HLA-A2 positive monocytes were dependent on the seeding density of the PBMCs.

### 3. Effects of Varying Seeding Density of HUVECs

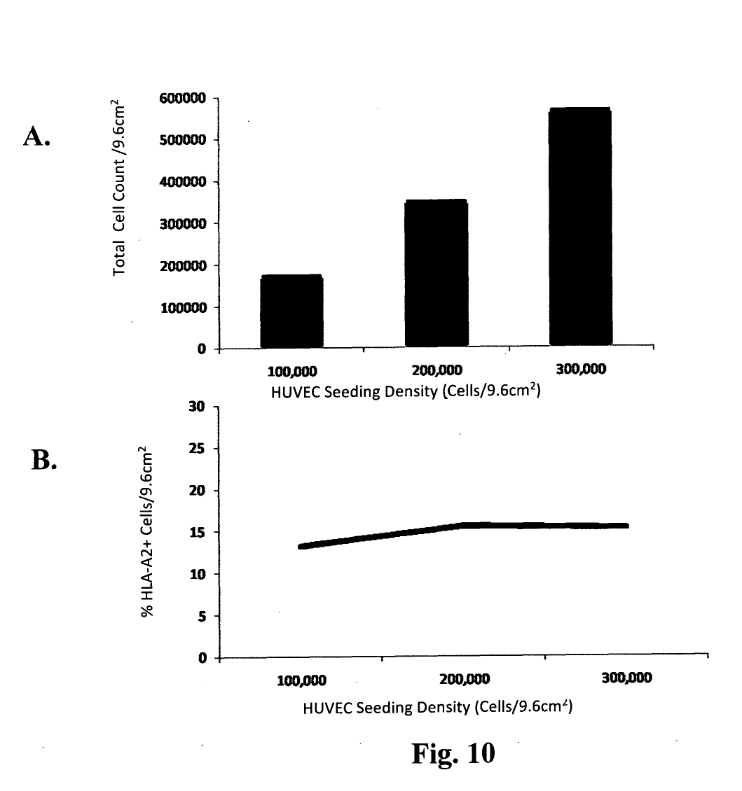

The inventor also investigated whether seeding density of the substrate endothelial cell affects the proportion of recruited monocytes that can trans-differentiated into EC-like cells, as observed by expression of cell surface markers during PBMC/HUVEC co-culture. HLA-A2 negative HUVECs were grown as described supra and cultured on 6-wells cell culture plates (Bectin Dickinson Labware, 9.6 cm² plating surface area/well) at densities of 0.1×10⁶ cells per well (low density), 0.2×10⁶ cells per well (intermediate density) and 0.3×10⁶ cells per well (high density). The HUVECs were plated 24 hours before the experiment from a single batch of HUVEC for each experiment. The cell layers were sub-confluent at the time of commencing the co-culture. The PBMC seeding density was 1×10⁶ cells per well. The total cell counts after 24 hours of co-culture were 0.17×10⁶ cells per well in the low density group, 0.35×10⁶ cells per well in the intermediate density group and 0.57×10⁶ cells in the high density group (FIG. 10A). The percentages of monocyte-derived EC-like cells recruited by the HUVEC and incorporated into the EC layer were remarkably consistent at 14.7±1.3% despite the varying HUVEC densities (FIG. 10B).

The inventor reasoned that the recruitment of monocyte being independent of endothelial density provide strong evidence of an active recruitment process of monocytes onto the EC layer. Should this be a passive settling of the monocytes onto the culture plate, the percentage of monocyte-derived EC-like cells would be expected to increase at the lower endothelial densities rather than remaining relatively constant.

These results provided herein demonstrate that the method of the invention provides a reliable index and/or measure for quantifying the ratio of monocyte-derived EC-like cells recruited onto the endothelial layer relative to the monocytes substrate in the culture and/or relative to the endothelial cell substrate in the culture.

### Example 5

### Lipid Free Apolipoprotein A-I Increased Endothelial Incorporation of Monocyte-Derived Endothelial-Like Cells

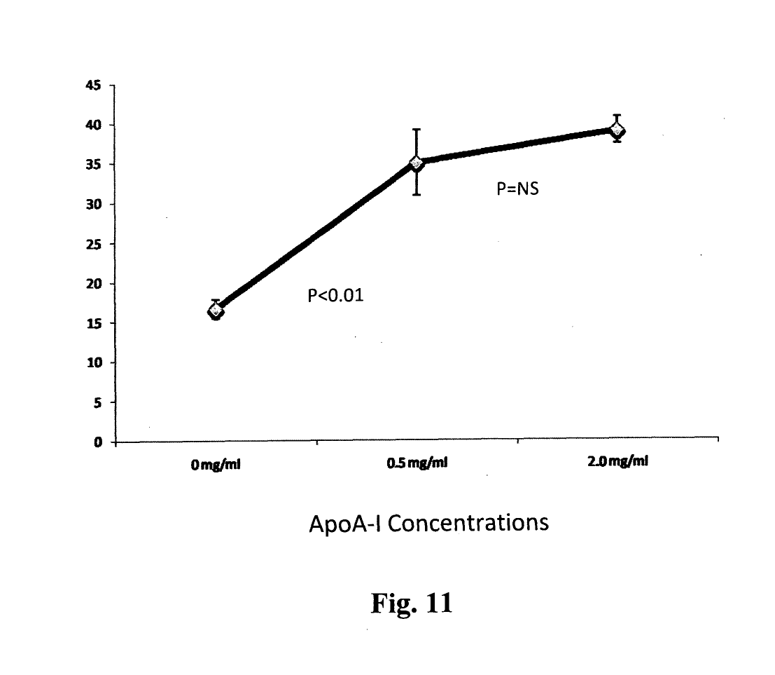

The inventor assessed the effect of lipid free apoA-I isolated from human plasma HDL on the incorporation of monocyte-derived endothelial-like cells into the endothelial layer (FIG. 11). As described supra, apoA-I was added to a co-culture of HLA-A2 positive monocytes and HLA-A2 negative HUVECs at concentrations of 0.25 mg/ml, 0.5 mg/ml and 2.0 mg/ml. A control to which no apoA-I had been added was analyzed. Percentages of adherent HLA-A2 positive cells of the total cells in the endothelial cell layer were analyzed on day 0 and day 3 following contact of the monocytes with the HUVECs layer in the co-culture. Day 3 results are shown in FIG. 11. The results illustrate a dose dependent increase in endothelial incorporation of HLA-A2 positive monocyte-derived endothelial-like cells into the HUVECs layer. Consistent with the results supra, no apoA-I control had about 15% monocyte-derived EC-cells incorporated into the HUVECs layer at day 3. However when cells were co-cultured in the presence of 0.5 mg/ml apoA-I the proportion of HLA-A2 positive cells increased dramatically to about 33% of the total cell population (t test P<0.01), this proportion was further increased to about 37% following incubation with 2.0 mg/ml apoA-I. These results indicate that apo-A-1 increases endothelial incorporation of monocyte-derived endothelial-like cells, implicating HDL in vascular repair.

### Example 6

### Lipid Free Apolipoprotein A-II Increases Endothelial Incorporation of Monocyte-Derived Endothelial-Like Cells

The inventor assesses the effect of lipid free apoA-II isolated form human plasma HDL on the incorporation of monocyte-derived endothelial-like cells into the endothelial layer. As described supra, apoA-II is added to a co-culture of HLA-A2 positive monocytes and HLA-A2 negative HUVECs at concentrations of 0.25 mg/ml and 0.4 mg/ml. A control co-culture to which no apoA-II is added is also analyzed. Percentages of adherent HLA-A2 positive cells of the total cells in the endothelial cell layer are analyzed on day 0 and day 5 following contact of the monocytes with the HUVECs layer in the co-culture. Results demonstrating an increase in endothelial incorporation of HLA-A2 positive monocyte-derived endothelial-like cells into the HUVECs layer in the presence of apoA-II, will support a role for HDL in vascular repair.

### Example 7

### LDL Had a Negative Effect on Endothelial Incorporation of Monocyte-Derived Endothelial-Like Cells

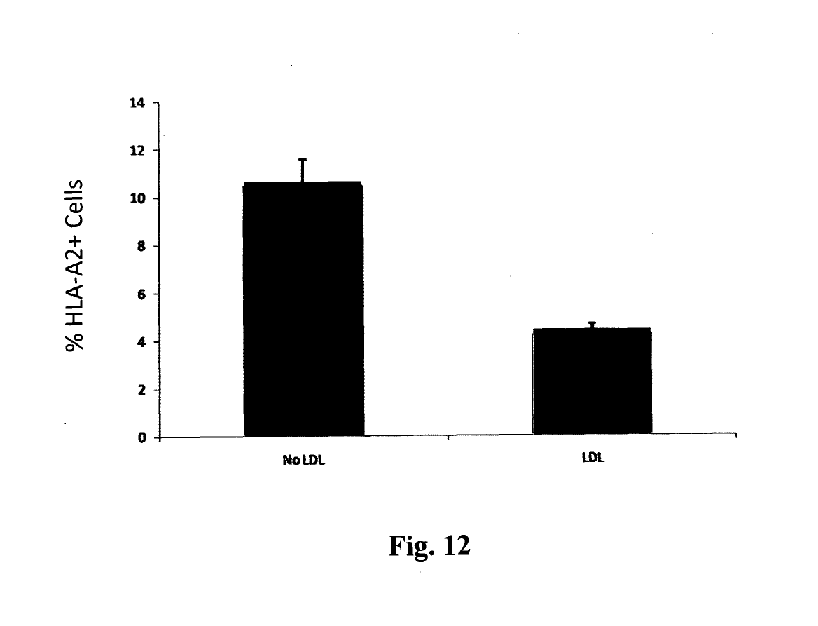

The inventor has assessed the effect human plasma LDL on the incorporation of monocyte-derived endothelial-like cells into the endothelial layer. Human LDL isolated from fresh human plasma was added to a test co-culture of HLA-A2 positive monocytes and HLA-A2 negative HUVECs at a concentration of 0.5 mg/ml, but not to a control co-culture of monocytes and HUVECs. Percentage of adherent HLA-A2 positive cells of the total cell count in the endothelial cell layer was analyzed on day 1 and day 2 following co-culture and shown in FIG. 12. The results indicate that the mean proportion of HLA-A2 positive cells (at both day 1 and day 2) that was incorporated into the HUVECs layer had significantly declined from about 10% of the total cell population when co-culturing cells in the absence of LDL to about 4% in presence of LDL (t test P<0.01). Therefore, LDL significantly antagonised the incorporation HLA-A2 positive adherent monocyte-derived EC-like cells into the endothelial layer following contact with the ECs, and thereby antagonised vascular repair processes.

### Example 8

### Monocyte-Derived Endothelial-Like Cells Participate in EC Formation of Vascular-Like Structure Following Incorporated into the Endothelium

To determine whether or not adherent monocyte-derived EC-like cells are capable of participating in endothelial repair, the ability of the monocyte-derived ECs to proliferate, migrate and participate in EC formation of vascular like structures is determined by in vitro angiogenic assays PBMCs and HUVECs or PBMCs and HACECs are co-cultured for 2 to 3 hrs followed by washing off the non-adherent cells and maintaining the co-culture up to 6 days, essentially as described in Examples 1 to 3 supra. Cells are characterized on day 1 (i.e., at 24 hours), day 2 (i.e. at 48 hours) and day 3 (i.e., at 72 hours) following commencement of co-culture, at which point cells are detached and HLA-A2 positive monocytes-derived EC-like cells expressing CD105, CD144 but not CD14 are sorted and assessed for cell proliferation by EdU incorporation, cell migration and tubulogenesis on growth-factor reduced Matrigel, essentially as described in Example 1 supra. As untreated substrate ECs control, density matched HLA-A2 negative HUVECs and HACECs expressing CD105 and CD144 but not CD14 seeded onto M199 media supplemented with 10% foetal bovine serum (FBS; Trace Bioscience) or with 10% heat-inactivated human serum and with a D-glucose concentration of 5.0 mM are used. As untreated substrate monocytes control, density matched HLA-A2 positive PBMCs expressing CD14 seeded onto M199 media supplemented with 10% foetal bovine serum (FBS; Trace Bioscience) or with 10% heat-inactivated human serum and with a D-glucose concentration of 5.0 mM are used.

In this example, a finding that HLA-A2 positive monocyte-derived EC-like cells expressing CD105, CD144 show proliferation profile, migration profile and EC formation of vascular like structures on Matrigel supports a conclusion that adherent monocyte-derived EC-like cells may participate in vascular generation e.g., by contributing to angiogenesis and/or vascularization.

### Example 9

### Apolipoprotein Enhances Formation of Vascular-Like Structures from Monocyte-Derived Non-Haematopoietic cells and LDL has a Negative Effect Thereon

To determine whether or not lipid-free apoA-1 and/or lipid-free apoA-II and/or apo-A-IV are capable of modulating proliferation, migration and tubulogenesis of monocyte-derived endothelial-like cells, the incorporation of EC-like cells into endothelium is determined in the presence and absence of apoA-I and/or apo-A-II and/or apo-AIV. LDL may be included in such assays as an antagonist.

For example, PBMCs and HUVECs or PBMCs and HACECs are co-cultured for 3 hrs followed by washing off non-adherent cells and maintaining the co-culture up to 6 days in the presence of apoA-I and/or apoA-II and/or apo-AIV, and LDL, essentially by the method described in Examples 5 to 7 hereof. Cells are characterized at day 0, day 1 (i.e., at 24 hours), day 2 (i.e. at 48 hours) and day 4 (i.e., at 96 hours) following commencement of co-culture, at which point cells are detached and HLA-A2 positive monocytes-derived EC-like cells expressing CD105, CD144 but not CD14 are sorted and assessed for cell proliferation by EdU incorporation, cell migration and tubulogenesis on growth-factor reduced Matrigel, essentially as described in Example 1 supra. Untreated co-culture of PBMCs and HUVECs or PBMCs and HACECs are subjected to the same culture conditions as the apolipoprotein-containing samples and LDL controls, albeit in the absence of these compounds.

In this example, a finding that HLA-A2 positive monocyte-derived HUVEC or HACEC EC-like cells expressing CD105, CD144 show enhanced proliferation profile, migration profile and EC formation of vascular like structures on Matrigel in the presence of apoA-I and/or apoA-II and/or apo-AIV compared to the absence thereof supports a conclusion that apoA-I and/or apo-A-II and/or apo-AIV enhances angiogenesis and/or neovascularization and therefor may be suitable for enhancing the vascular generation processes.

### Example 10

### Diagnosis of Atherosclerosis or a Predisposition for Atherosclerosis

The example demonstrates the utility of an assay of the present invention to assess atherosclerosis or a predisposition for atherosclerosis in a subject, wherein monocytes from a subject are assayed for their ability to differentiate into EC or EC-like cells and preferably the ability of the EC or EC-like cells produced to incorporate into endothelium.

Venous blood samples are obtained from HLA-A2 positive subjects with confirmed vascular complications and atherosclerosis. Atherosclerotic patients are recruited with informed consent and have their clinical, biochemical and haematological data collected. PBMCs are obtained from the venous blood samples collected by gradient centrifugation on Lymphoprep as previously described in Example 1 and immediately seeded onto HUVECs or HCAECs. PBMCs and HUVECs or PBMCs and HACECs are co-cultured for 3 hrs followed by washing to remove non-adherent cells and maintaining the co-culture up to 6 days for co-culturing as described in Examples 1 to 3. Cells are characterized at day 1, day 2 and day 3 following commencement of co-culture. Co-cultures of HUVECs or HCAECs with PBMCs obtained from venous blood of sex and age matched healthy HLA-A2 donors as previously described, are used as controls. Production of HLA-A2 positive cells expressing CD105, CD144 but not CD14 in each co-culture is determined by flow cytometry and cells are also sorted and analyzed for proliferation, migration and tubulogenesis in vitro angiogenesis assays as described supra, and the groups are compared.

In this diagnostic assay format, monocytes from subjects having atherosclerosis or vascular complications or a predisposition thereto will produce a lower proportion of adherent HLA-A2 positive EC-like cells expressing CD105, CD144 but not CD14 than monocytes from healthy subjects. Similarly, EC or EC-like cells derived from monocytes of subjects having atherosclerosis or vascular complications or a predisposition thereto will have low rates of proliferation, migration or EC formation of vascular like structures as determined on matrigel compared to EC or EC-like cells derived from monocytes of healthy subjects.

### Example 11

### Diagnosis of Atherosclerosis or a Predisposition for Atherosclerosis

This example demonstrates the utility of an assay of the invention to diagnose atherosclerosis or a vascular complication or a predisposition thereto in a subject, wherein the assay sample comprises a monocyte-depleted fraction of blood and wherein the sample is assayed for an ability to modulate the ability of exogenous monocytes to differentiate into EC or EC-like cells and preferably to modulate the ability of the EC or EC-like cells to incorporate into endothelium.

Pooled whole venous blood samples obtained from HLA-A2 positive subjects with confirmed vascular complications and atherosclerosis (described in Example 10), and pooled whole venous blood samples obtained form sex and age matched healthy subjects (Example 1) are depleted of monocytes using the RosetteSep® procedure (StemCell Technologies) according to manufacturer's instructions. Monocyte-depleted plasma fractions are collected. 100 μl, 300 μl, 500 μl and 1 ml aliquots of each monocyte-depleted plasma fraction are added to a co-culture of adherent HLA-A2 positive monocytes obtained from healthy subjects and HLA-A2 negative HUVECs or HLA-A2 negative HACECs (as described in Example 1). Proportions of adherent HLA-A2 positive cells expressing CD105, CD144 but not CD14 are analyzed on day 1, day 2 and day 3 following commencement of co-culture, and the groups are compared. A control co-culture to which no plasma fraction is added but which receives equal volumes of sterile PBS is also analyzed. Analyses demonstrate that PBMCs/HUVECs or PBMC/HACECs co-cultures to which Monocyte-depleted plasma fraction from atherosclerotic subjects are added, produce reduced levels of monocyte-derived HLA-A2 positive EC-like cells expressing CD105, CD144 but not CD14 compared with co-cultures containing plasma fraction from healthy subjects, or compared with no plasma controls.

Also, HLA-A2 positive cells expressing CD105, CD144 but not CD14 from each co-culture are sorted and analyzed for proliferation, migration and tubulogenesis in vitro angiogenesis assays as described supra, and the groups are compared.

In this diagnostic assay format, the ability of a monocyte-depleted assay sample to reduce differentiation of monocytes to EC or EC-like cells and/or to reduce the incorporation of EC or EC-like cells into endothelium is inductive of atherosclerosis or vascular complication or a predisposition thereto. Reduced differentiation and/or incorporation will be apparent when compared to the level of differentiation and/or incorporation observed for healthy control samples, and may approach a level or be consistent with a level observed for samples from atherosclerosis subjects.

### Example 12

### Apolipoprotein A Alleviates Antagonism of Monocyte Differentiation into ECs or EC-Like Cells by Plasma Fractions from Atherosclerotic Subjects

This example demonstrates utility of apoA-I and/or apoA-II and/or apoA-IV in therapy of atherosclerosis or vascular complication associated therewith by virtue of agonizing differentiation of monocytes into EC or EC-like cells and/or agonizing the incorporation of EC or EC-like cells into endothelium.

Monocyte-depleted plasma fraction from subjects with vascular complications and atherosclerosis are added, in 100 μl, 300 μl, 500 μl and 1 ml aliquots to a co-culture of adherent HLA-A2 positive monocytes obtained from healthy subjects and HLA-A2 negative HUVECs or HLA-A2 negative HACECs (as described in Example 11). apoA-I and/or apoA-II and/or apoA-IV is(are) added to each co-culture at concentrations 0.5 mg/ml and 2.0 mg/ml. Proportions of adherent HLA-A2 positive cells expressing CD105, CD144 but not CD14 are analyzed on day 1, day 2 and day 3 following commencement of co-culture, and the groups are compared. A control to which no apolipoprotein is added is also analyzed.

HLA-A2 positive cells expressing CD105, CD144 but not CD14 in each co-culture also sorted and analyzed for proliferation, migration and tubulogenesis in vitro angiogenesis assays as described supra, and the groups are compared.

In this example, the ability of apoA-I and/or apoA-II and/or apoA-IV to promote monocyte differentiation into EC or EC-like cells, or to alleviate or reverse antagonism of monocyte differentiation by monocyte-depleted plasma from atherosclerotic subjects is indicative of therapeutic efficacy. Similarly, the ability of apoA-I and/or apoA-II and/or apoA-IV to promote incorporation of EC or EC-like cells derived from monocytes into endothelium, or to alleviate or reverse antagonism of such incorporation by monocyte-depleted plasma from atherosclerosis subjects, is indicative of therapeutic efficacy.

1. A method of differentiating monocytes into non-haematopoietic cells, said method comprising contacting substrate monocytes with substrate ECs for a time and under conditions sufficient for non-haematopoietic cells to be produced from the substrate monocytes.

2. The method according to claim 1, wherein the produced non-haematopoietic cells are ECs or endothelial-like cells expressing CD144 and/or CD105.

3. The method according to claim 1 or 2, wherein the substrate monocytes and produced non-haematopoietic cells express the same alleles of a detectable marker and wherein substrate ECs express one or more different alleles of the detectable marker to thereby distinguish the substrate ECs from the substrate monocytes and from the produced non-haematopoietic cells.

4. The method according to claim 3, further comprising determining expression of one or more different alleles of the detectable marker in the substrate monocytes and/or the produced non-haematopoietic cells and/or the substrate ECs, wherein expression of different alleles of the detectable marker in the substrate ECs to the alleles of the detectable marker expressed by the substrate monocytes and produced non-haematopoietic cells is indicative of non-haematopoietic cell production from the monocytes.

5. The method according to claim 4, comprising determining:
(i) expression of the detectable marker in the substrate monocytes and the substrate ECs; or
(ii) expression of the detectable marker in the substrate ECs and in the produced non-haematopoietic cells; or
(iii) expression of the detectable marker in the substrate monocytes and in the substrate ECs and in the produced non-haematopoietic cells.

6-7. (canceled)

8. The method according to claim 3, wherein the detectable marker is a cell surface antigen or a human leukocyte antigen.

9. (canceled)

10. The method according to claim 1, further comprising quantitating production of non-haematopoietic cells from substrate monocytes.

11. The method according to claim 10, wherein quantitating production of non-haematopoietic cells from substrate monocytes comprises determining a ratio or percentage or number of substrate monocytes that differentiate into non-haematopoietic cells.

12. The method according to claim 11, wherein substrate monocytes and/or produced non-haematopoietic cells are determined by the presence or absence of one or more surface cell-type specific markers.

13. The method according to claim 12, wherein a surface cell-type specific marker is a cluster designation (CD) marker.

14. The method according to claim 12, wherein a marker is selected from the group consisting of CD14, CD11b, CD36, CD115, CD34, CD105, CD133, CD144, Tie-2 KDR, ICAM-1 and VCAM-1.

15. The method according to claim 1, wherein a time sufficient for non-haematopoietic cells to be produced is less than about 4 days.

16. The method according to claim 1 for determining the ability of a composition to produce non-haematopoietic cells or to promote the production of non-haematopoietic cells or to antagonize the production of non-haematopoietic cells.

17. The method according to claim 16 for determining the ability of a composition to produce non-haematopoietic cells, wherein the composition comprises substrate monocytes and wherein the method comprises determining production of non-haematopoietic cells from the substrate monocytes in the composition.

18. The method according to claim 16 for determining the ability of a composition to promote the production of non-haematopoietic cells, wherein the composition lacks monocytes or is monocyte-depleted and wherein the method comprises contacting exogenous substrate monocytes with the substrate ECs in the presence and absence of the composition for a time and under conditions sufficient for non-haematopoietic cells to be produced from the substrate monocytes and comparing non-haematopoietic cell production, wherein enhanced non-haematopoietic cell production in the presence of the composition indicates that the composition promotes the production of non-haematopoietic cells.

19. The method according to claim 16 for determining the ability of a composition to antagonize the production of non-haematopoietic cells, wherein the composition lacks monocytes or is monocyte-depleted and wherein the method comprises contacting exogenous substrate monocytes with the substrate ECs in the presence and absence of the composition for a time and under conditions sufficient for non-haematopoietic cells to be produced from the substrate monocytes and comparing non-haematopoietic cell production, wherein reduced non-haematopoietic cell production in the presence of the composition indicates that the composition antagonizes the production of non-haematopoietic cells.

20. The method according to claim 1 in screening for, isolating and/or identifying a modulator of angiogenesis and/or neovascularisation and/or atherogenesis and/or a vascular complication.

21. The method according to claim 1 for determining the likelihood of a subject having one or more vascular complications and/or atherosclerosis and/or at risk of developing one or more vascular complications and/or atherosclerosis wherein said method is performed in the presence of a sample obtained from the subject.

22. The method according to claim 1 for monitoring therapy of a subject for one or more vascular complications and/or atherosclerosis, wherein said method is performed in the presence of two or more samples obtained from the subject at different time points.

23. A process for determining the ability of a composition to modulate the production of non-haematopoietic cells from monocytes, said process comprising performing the method of claim 1 in the presence of a candidate modulatory composition to thereby determine the effect of the candidate modulatory composition on non-haematopoietic cell production and identifying and/or isolating a composition that promotes the production of non-haematopoietic cells or antagonizes the production of non-haematopoietic cells.

24. The process according to claim 23 performed in vivo.

25. The process according to claim 24, comprising administering a candidate modulatory composition to an animal subject and determining the effect of the composition on atherogenesis and/or one or more vascular maintenance and/or repair processes in the animal subject and identifying and/or isolating a composition that promotes or antagonizes atherogenesis and/or one or more vascular maintenance and/or repair processes in the animal subject.

26. The process according to claim 23, performed in vitro.

27. The process according to claim 23, wherein the candidate modulatory composition is an agonist or partial agonist of non-haematopoietic cell production.

28. The process according to claim 27, further comprising performing the method in the absence of substrate ECs and comparing the effect of the agonist or partial agonist in the presence and absence of substrate ECs.

29. The process according to claim 28, further comprising identifying and/or isolating a composition that promotes the production of non-haematopoietic cells in the absence of substrate ECs.

30. The process according to claim 23, wherein the candidate modulatory composition is an antagonist of non-haematopoietic cell production.

31. The process according to claim 30, further comprising performing the method in the presence of an agonist of endothelial cell production and comparing the effect of the antagonist on non-haematopoietic cell production in the presence and absence of the agonist.

32. The process according to claim 31, wherein the agonist of endothelial cell production comprises fibronectin and one or more growth factors under angiogenic culture conditions.

33. The process according to claim 31, performed in a single reaction in the presence of the agonist.

34. The process according to claim 31, performed in separate reactions in the presence and absence of the agonist.

35. The process according to claim 31, further comprising identifying and/or isolating a composition that antagonizes the production of non-haematopoietic cells in the presence of the agonist.

36. The process according to claim 23, further comprising performing the method in the absence of the candidate modulatory composition and comparing non-haematopoietic cell production in the presence and absence of the candidate modulatory composition.

37. The process according to claim 23, wherein the candidate modulatory composition comprises a protein, polypeptide, peptide, antibody, nucleic acid, or small molecule.

38. The process according to claim 23, further comprising administering the isolated or identified composition to an animal subject and determining the effect of the composition on one or more vascular maintenance and/or repair processes in the animal subject.

39-46. (canceled)

47. A process of determining the likelihood of a subject having one or more vascular complications and/or atherosclerosis and/or at risk of developing one or more vascular complications and/or atherosclerosis, said process comprising performing the method according to claim 2 in the presence of a biological sample isolated from a subject, wherein production of ECs from substrate monocytes in the presence of the biological sample indicates that the subject has a reduced or low likelihood of having said one or more vascular complications and/or atherosclerosis and/or is not at a risk of having or developing said one or more vascular complications and/or atherosclerosis, and/or wherein non-production or low production of ECs from substrate monocytes in the presence of the biological sample indicates that the subject has one or more vascular complications and/or atherosclerosis and/or is at a risk of having or developing said one or more vascular complications and/or atherosclerosis.

48. The process according to claim 47, further comprising obtaining a sample from a subject.

49. The process according to claim 47, wherein the biological sample comprises the substrate monocytes and wherein the method comprises determining the ability of the substrate monocytes to differentiate into ECs, and wherein said differentiation indicates that the subject has a reduced or low likelihood of having said one or more vascular complications and/or atherosclerosis and/or is not at a risk of having or developing said one or more vascular complications and/or atherosclerosis, and/or wherein non-differentiation or a low efficiency of differentiation of ECs from substrate monocytes in the presence of the biological sample indicates that the subject has one or more vascular complications and/or atherosclerosis and/or is at a risk of having or developing said one or more vascular complications and/or atherosclerosis.

50. The process according to claim 49, wherein the sample comprises peripheral blood, umbilical cord blood, a fraction of peripheral blood comprising monocytes or a fraction of umbilical cord blood comprising monocytes.

51. The process according to claim 50, wherein a fraction of peripheral blood or a fraction of umbilical cord blood comprises plasma or fresh frozen plasma.

52. The process according to claim 47, wherein the sample lacks monocytes or is depleted of monocytes and wherein said process comprises contacting the sample with substrate monocytes and determining the ability of the sample to promote differentiation of the substrate monocytes into ECs, wherein said differentiation indicates that the subject has a reduced or low likelihood of having said one or more vascular complications and/or atherosclerosis and/or is not at a risk of having or developing said one or more vascular complications and/or atherosclerosis, and/or wherein non-differentiation or a low efficiency of differentiation of ECs from substrate monocytes in the presence of the biological sample indicates that the subject has one or more vascular complications and/or atherosclerosis and/or is at a risk of having or developing said one or more vascular complications and/or atherosclerosis.

53. The process according to claim 52, wherein the sample lacking monocytes or depleted of monocytes is autologous to the substrate monocytes.

54. The process according to claim 52, wherein the substrate monocytes are heterologous to the sample.

55. The process according to claim 52, further comprising depleting monocytes from a monocyte-containing sample.

56. The process according to claim 55, comprising depleting monocytes from the sample by a process comprising:
(i) performing density gradient centrifugation to thereby separate monocytes from plasma and retaining the plasma fraction; or
(ii) contacting the sample with a solid phase surface to thereby adhere monocytes and then retaining the unbound liquid fraction; and/or
(iii) contacting the sample with an antibody that binds CD14 for a time and under conditions sufficient to bind to CD14 on monocytes and then removing monocytes.

57-59. (canceled)

60. The process according to claim 56, wherein the antibody that binds CD14 is immobilized on a solid substrate.

61. The process according to claim 60, wherein the solid substrate comprises a particle.

62. The process according to claim 55, wherein the monocyte-containing sample comprises peripheral blood, umbilical cord blood, a fraction of peripheral blood comprising monocytes or a fraction of umbilical cord blood comprising monocytes.

63. The process according to claim 62, wherein a fraction of peripheral blood or a fraction of umbilical cord blood comprises plasma or fresh frozen plasma.

64. A process of determining the likelihood of a subject having one or more vascular complications and/or atherosclerosis and/or at risk of developing one or more vascular complications and/or atherosclerosis, said process comprising performing the method according to claim 2 in the presence of a biological sample isolated from a subject to thereby produce ECs and determining the ability of the produced ECs to incorporate into vascular tissue, wherein incorporation of ECs into vascular tissue indicates that the subject has a reduced or low likelihood of having said one or more vascular complications and/or atherosclerosis and/or is not at a risk of having or developing said one or more vascular complications and/or atherosclerosis, and/or wherein non-incorporation or low efficiency of incorporation of ECs into vascular tissue indicates that the subject has one or more vascular complications and/or atherosclerosis and/or is at a risk of having or developing said one or more vascular complications and/or atherosclerosis.

65. The process according to claim 64, further comprising obtaining the biological sample from the subject.

66. The process according to claim 64, wherein the biological sample comprises substrate monocytes and the production of ECs from said substrate monocytes is determined.

67. The process according to claim 64, wherein the sample lacks monocytes or is depleted of monocytes and wherein the production of ECs from substrate monocytes is determined in the presence of the sample.

68. The process according to claim 67, wherein the sample and substrate monocytes are autologous and/or the sample and produced ECs are autologous.

69. The process according to claim 64, wherein the ability of the produced ECs to incorporate into vascular tissue is determined by migration of ECs, proliferation of ECs, tubulogenesis of ECs, and combinations thereof.

70. The process according to claim 69 wherein the ability of the produced ECs to incorporate into vascular tissue is determined by Matrigel assay.

71. The process according to claim 64, further comprising determining the ability of the produced ECs to incorporate into vascular tissue in the presence of an athero-protective compound

72. The process according to claim 64, comprising determining the ability of the produced ECs to incorporate into vascular tissue in the presence and absence of an athero-protective compound.

73. The process according to claim 72, further comprising comparing the incorporation of the produced ECs into vascular tissue in the presence and absence of the athero-protective compound, wherein incorporation in the presence but not absence of the athero-protective compound or enhanced incorporation in the presence of the athero-protective compound relative to incorporation in the absence of the athero-protective compound indicates that the subject is likely to have one or more vascular complications and/or atherosclerosis and/or is at a risk of having or developing said one or more vascular complications and/or atherosclerosis.

74. The process according to claim 71, wherein the athero-protective compound comprises statin and/or a high-density lipoprotein (HDL) and/or a compound that mimics a HDL and/or apolipoprotein A-I and/or apolipoprotein A-II.

75-78. (canceled)

79. The process according to claim 65, wherein the sample comprises plasma, serum, a fraction of plasma or a fraction of serum.

80-90. (canceled)

91. A process of monitoring vascular therapy of one or more vascular complications and/or atherosclerosis in a subject, said process comprising performing the method of claim 2 in the presence of two or more biological samples from a subject at different time points, wherein enhanced production of ECs from substrate monocytes in the presence of a sample obtained at a later time point indicates that the subject has responded to vascular therapy.

92. The process according to claim 91, wherein at least one of the two or more samples is obtained from the subject prior to vascular therapy and at least one of the two or more samples is obtained from the subject after commencement of vascular therapy and wherein enhanced endothelial cell production by a sample obtained from the subject after commencement of vascular therapy indicates effective therapy.

93. A process of monitoring vascular therapy of one or more vascular complications and/or atherosclerosis in a subject, said process comprising performing the method according to claim 2 in the presence of two or more biological samples from a subject at different time points to thereby produce two or more batches of ECs, and determining the ability of each of said two or more batches of ECs to incorporate into vascular tissue, wherein enhanced incorporation of ECs into vascular tissue in the presence of a sample obtained at a later time point indicates that the subject has responded to vascular therapy.

94. The process according to claim 93, wherein at least one of the two or more samples is obtained from the subject prior to vascular therapy and at least one of the two or more samples is obtained from the subject after commencement of vascular therapy and wherein enhanced endothelial cell production by a sample obtained from the subject after commencement of vascular therapy indicates effective therapy.

95-96. (canceled)

97. A process for treatment of one or more vascular complications and/or atherosclerosis in a subject, said process comprising performing the process according to claim 47 and administering or recommending therapy effective to alleviate one or more vascular complications and/or atherosclerosis to a subject in need thereof.

98. A process for treatment of one or more vascular complications and/or atherosclerosis in a subject, said process comprising performing the process according to claim 64 and administering or recommending therapy effective to alleviate one or more vascular complications and/or atherosclerosis to a subject in need thereof.

99. (canceled)

100. A process for treatment of one or more vascular complications and/or atherosclerosis in a subject, said process comprising performing the process according to claim 91 and administering or recommending therapy effective to alleviate one or more vascular complications and/or atherosclerosis to a subject in need thereof.

101. A process for the treatment of one or more vascular complications and/or atherosclerosis in a subject, said process comprising performing the process according to claim 93 and administering or recommending therapy effective to alleviate one or more vascular complications and/or atherosclerosis to a subject in need thereof.

102-108. (canceled)
